(19) United States
(12) Patent Application Publication
(10) Pub. No.: US 2026/0276614 A1
(43) Pub. Date: Sep. 17, 2026

(54) Synthesis of Air Quality Data from Diverse Sources Using Machine Learning

(71) Applicant: The Lution Company, Ojai, CA (US)

(72) Inventors: David A. Vivero, Los Angeles, CA (US); Michael Marino, Ojai, CA (US)

(73) Assignee: The Lution Company, Ojai, CA (US)

(21) Appl. No.: 19/080,716

(22) Filed: Mar. 14, 2025

(51) Int. Cl. G01N 33/00; G06N 20/00

(52) U.S. Cl. CPC G01N 33/0063; G06N 20/00

(57) ABSTRACT

Composite health impact scoring of air quality for multiple pollutants is described. In one or more implementations, input data is received indicating concentrations of multiple pollutants at a geographic location. A composite health impact score is generated based on the input data. As part of this, response factors are applied to the multiple pollutants, where the response factors quantify health impacts from exposure to respective pollutants of the multiple pollutants. Moreover, independence factors are applied to the multiple pollutants, where the independence factors adjust the health impacts attributable to the respective pollutants based on combined exposure to the multiple pollutants. The composite health impact score is communicated to a client device for output in a user interface.

### BACKGROUND

Air quality monitoring involves the continuous measurement and analysis of pollutants and environmental parameters to assess the composition and cleanliness of the air we breathe. It plays a vital role in identifying harmful contaminants such as particulate matter (PM2.5 and PM10), ozone (O₃), nitrogen dioxide (NO₂), sulfur dioxide (SO₂), and carbon monoxide (CO), which can adversely affect human health. Monitoring systems—ranging from large regulatory stations to compact IoT sensors—collect data on pollutant concentrations to evaluate local and regional air quality trends. This information is critical for understanding health impacts based on the air that human beings breathe.

### SUMMARY

Composite health impact scoring of air quality for multiple pollutants is described. As part of this, input data is received indicating concentrations of multiple pollutants at a geographic location, exposure patterns to the multiple pollutants at the geographic location, a plurality of local pollution sources within a radius of the geographic location associated with pollution emission metrics, and local meteorological and topographical conditions at the geographic location that impact pollutant dispersion. A composite health impact score is generated based on the input data.

To generate the composite health impact score, response factors are applied to the multiple pollutants, where the response factors quantify health impacts from exposure to respective pollutants of the multiple pollutants. Moreover, independence factors are applied to the multiple pollutants, where the independence factors adjust the health impacts attributable to the respective pollutants based on combined exposure to the multiple pollutants. In addition, temporal weighting factors are applied to the multiple pollutants, where the temporal weighting factors account for varying health impacts derived from the exposure patterns. In some implementations, an atmospheric amplification factor is applied to the multiple pollutants based on the local meteorological and topographical conditions at the geographic location that impact pollutant dispersion. Additionally or alternatively, the composite health impact score is modified based on the pollution emission metrics from the plurality of local pollution sources. The modified composite health impact score is communicated to a client device for output in a user interface.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ techniques described herein.

FIG. 2 depicts a system in an example implementation showing operation of an air quality analysis system to leverage a machine learning model during a pre-processing phase for composite health impact scoring of air quality for multiple pollutants.

FIG. 3 depicts a system in an example implementation showing operation of a health impact scoring module to leverage a deterministic algorithm during a runtime processing phase for composite health impact scoring of outdoor air quality for multiple pollutants.

FIG. 4 depicts a system in an example implementation showing operation of a health impact scoring module to leverage a deterministic algorithm during a runtime processing phase for composite health impact scoring of indoor air quality for multiple pollutants.

FIG. 5 depicts an example user interface displaying composite health impact scores.

FIG. 6 depicts an example user interface displaying local pollution sources and composite health impact scores attributable to health domains.

FIG. 7 depicts an example user interface displaying a summary of atmospheric factors and detected pollutants.

FIG. 8 depicts an example user interface showing cancer-causing chemicals detected near a geographic location being evaluated for air quality.

FIG. 9 depicts an example user interface showing terrain data contributing to a composite health impact score at a geographic location.

FIG. 10 depicts an example user interface showing traffic volume data contributing to a composite health impact score at a geographic location.

FIG. 11 depicts an example user interface showing registered emitters within a radius of a geographic location.

FIG. 12 depicts an example user interface showing local pollution sources within a radius of a geographic location.

FIG. 13 depicts an example user interface showing an air quality history at a geographic location.

FIG. 14 is a flow diagram depicting an algorithm as a procedure in an example implementation that is performable by at least one processing device.

FIG. 15 illustrates an example of a system that may implement the various techniques described herein.

### DETAILED DESCRIPTION

### Overview

Conventional air quality assessment methods suffer from significant limitations that prevent accurate evaluation of cumulative health impacts from multiple pollutant exposures. Traditional approaches, such as the Environmental Protection Agency's Air Quality Index (AQI), rely on single-pollutant maximum values and linear relationships that only consider the highest individual pollutant at any given time. This traditional approach ignores the combined effects of multiple pollutants that could be contributing to adverse health outcomes. For example, three different environments—a rural agricultural valley, a suburban area with fireplace emissions, and an urban traffic corridor—may all receive identical AQI scores of 35 based on similar PM2.5 levels, despite the urban traffic corridor containing a dangerous mix of additional pollutants like sulfur dioxide, nitrogen dioxide, and ozone that significantly increases actual health risks. This single-pollutant approach fails to capture the reality that people are simultaneously exposed to multiple pollutants that interact through shared biological pathways and create synergistic health effects.

Furthermore, existing air quality monitoring systems are limited by their reliance on point-in-time measurements rather than temporal pattern analysis, which fails to account for the varying health impacts derived from temporal exposure patterns to various pollutants. Current methods do not distinguish between consistent moderate exposure and intermittent severe spikes, even though two individuals with identical average particulate matter exposure may experience vastly different health outcomes if one has steady moderate levels while the other experiences mostly clean air punctuated by monthly severe concentration spikes.

To address these limitations, composite health impact scoring of air quality for multiple pollutants is described. As part of this, an air quality analysis system prompts a machine learning model (e.g., a large language model) to analyze a compendium of scientific studies. For example, the compendium of scientific studies includes peer-reviewed research papers, epidemiological studies, toxicological assessments, and health impact analyses that document relationships between pollutant exposure and human health outcomes, including dose-response analyses, meta-analyses, and research on synergistic effects between multiple pollutants. The air quality analysis system instructs the machine learning model to analyze the compendium of studies to produce specific outputs.

The outputs produced by the machine learning model include an independence factor matrix, response factors for various pollutants, and one or more deterministic algorithms for determining a composite health impact score quantifying health impacts from exposure to multiple pollutants at a specific geographic location. Generally, the independence factor matrix is a structured table with the same set of pollutants along both axes, where individual cells contain independence factors quantifying the interaction effects between specific pollutant pairs. Independence factors are quantitative coefficients that prevent overestimation of cumulative health impacts by accounting for overlapping biological pathways and synergistic effects when multiple pollutants are present simultaneously. The response factors are quantitative coefficients that translate pollutant concentrations into health impact metrics, with each response factor being specific to a particular pollutant and quantifying the expected health impact per unit of exposure.

To generate an outdoor health impact score, the air quality analysis system receives a variety of input data from one or more online data sources. This input data may be received through application programming interfaces (APIs) that facilitate communication of the input data between the air quality analysis systema and the online data sources. The input data includes an outdoor air data history, terrain data, and local pollution sources. The outdoor air data history includes time-series data representing a comprehensive record of pollutant concentrations at various points in time (e.g., daily, hourly) over a duration of evaluation (e.g., two years) at the geographic location to capture exposure patterns that describe temporal variations in the pollutant concentrations. The terrain data comprises geographical and topographical information about the physical landscape surrounding the geographic location, including elevation profiles and natural barriers that influence air flow patterns and pollutant dispersion. The local pollution sources include facilities and establishments within a specified radius of the geographic location that emit pollutants, where the local pollution sources are associated with emission metrics that quantify pollutant output.

Given a pollutant indicated by the outdoor air data history as present at the geographic location, the deterministic algorithm(s) applies the pollutant's response factor to its baseline concentration. For example, the pollutant's response factor is multiplied by its baseline concentration (e.g., the average concentration over the duration of evaluation) to generate an individual health impact score for the pollutant. In some examples, the baseline concentration of a pollutant is a relative measurement of the amount by which the average concentration of the pollutant exceeds a pristine air baseline. Notably, the pristine air baseline of a pollutant represents pre-industrialization exposure levels of the pollutant.

The deterministic algorithm(s) further apply an independence factor to the individual health impact score. The pollutant's independence factor is extracted from the independence factor matrix. The pollutants detected at the geographic location include a primary contributor and secondary contributors. The primary contributor is a detected pollutant with the highest concentration relative to its pristine air baseline. Secondary contributors are the remaining pollutants that contribute to the overall health impact but at lower relative concentrations compared to the primary contributor.

An independence factor for a pollutant is extracted from a cell in the independence factor matrix corresponding to the row-column combination of that pollutant and the primary contributor. Thus, if the pollutant is the primary contributor, the independence factor is one, e.g., the cell corresponding to the row-column combination of the primary contributor with itself. If, however, the pollutant is a secondary contributor, the independence factor is extracted from the row-column combination of the secondary contributor with the primary contributor. The value of the independence factor for a secondary contributor is between zero and one to represent the synergistic health impacts of the secondary contributor when simultaneously exposed to humans with the primary contributor. In some examples, the independence factors assigned to secondary contributors are weighted based on concentrations of the secondary contributors relative to their pristine air baseline.

The deterministic algorithm(s) apply a temporal weighting factor to the pollutant's individual health impact score, where the temporal weighting factor is derived from the exposure patterns of the pollutant. The temporal weighting factor represents a quantitative coefficient that adjusts health impact calculations based on the timing, duration, and intensity patterns of pollutant exposure over time. The deterministic algorithm(s) determine the temporal weighting factor by analyzing the exposure patterns of the pollutant, such as by calculating variance in pollutant concentrations over the evaluation period, identifying frequency and magnitude of concentration spikes above baseline levels, and determining duration of elevated exposure periods. In some examples, since short bursts of elevated exposure may cause greater health impacts than sustained levels of lower exposure, the system may assign higher coefficients to pollutants with greater concentration variability and more frequent exposure spikes.

In one or more implementations, the deterministic algorithm(s) apply an atmospheric amplification factor the pollutant's individual health impact score. The deterministic algorithm(s) determine the atmospheric amplification factor by analyzing terrain data including elevation profiles, valley configurations, coastal proximity, urban canyon effects, prevailing winds (including direction and magnitude), and natural barriers, then calculating how these topographical features impact air circulation and pollutant retention at the geographic location.

This computation is repeated to generate an individual health impact score for each of the detected pollutants, and the individual health impact scores are aggregated across the detected pollutants. In one or more implementations, a local pollution source modifier is applied to the aggregation result, where the local pollution source modifier is based on the emission metrics of the local pollution sources within a radius of the geographic location. To do so, deterministic algorithm(s) calculate distance-weighted contributions for the local pollution sources based on their associated emission metrics and proximity to the geographic location, with closer pollution sources receiving higher weights. The deterministic algorithm(s) aggregate these contributions into a local pollution source modifier that reflects the cumulative impact of the local pollution sources. As a result, an outdoor composite health impact score is generated that quantifies the cumulative health impact from exposure to multiple pollutants. In various examples, the outdoor composite health impact score is communicated to a client device for output.

In contrast to conventional air quality assessment methods that rely on single-pollutant maximum values and linear relationships, the described composite health impact scoring system implements a sophisticated multi-dimensional approach that addresses the fundamental limitations of traditional techniques. As previously mentioned, conventional methods like the EPA's Air Quality Index only consider the highest individual pollutant at any given time and ignore the combined effects of multiple pollutants. In contrast, the described system leverages independence factors that account for the synergistic effects of combined exposure to multiple pollutants while reducing overestimation by accounting for overlapping biological pathways impacted by the multiple pollutants. Furthermore, the temporal weighting factors capture the varying health impacts derived from different exposure patterns over time rather than relying on point-in-time measurements. The described solution grounds the score in human health impact by applying response factors to specifically quantify how the prevailing conditions of air at the geographic location impact human health. Unlike conventional solutions, the system incorporates atmospheric amplification factors that adjust pollutant concentration measurements based on local meteorological and topographical conditions that influence pollutant dispersion, and local pollution source modifiers that account for proximity-based pollution exposure from nearby emission sources that may not be captured in regional air quality data. Furthermore, by measuring health impacts relative to pristine air baselines, the described system provides users with a more meaningful assessment of actual health consequences from air pollution exposure. These features of the described solution provide a more comprehensive and accurate assessment of human health impacts from exposure to multiple pollutants than conventional methods.

The described system's two-stage processing approach reduces runtime processing latency and enables real-time or near real-time score generation by separating the resource-intensive analysis of scientific literature from time-sensitive runtime computations. During the pre-processing stage, machine learning models thoroughly analyze compendiums of scientific studies to generate optimized deterministic algorithms that encode complex relationships between pollutants and health impacts At runtime, the system executes these pre-computed deterministic algorithms to achieve faster response times and reduced computational overhead compared to performing resource-intensive machine learning inference for each user query. This approach enables real-time or near-real-time composite health impact scoring while minimizing processing latency, memory usage, and energy consumption during user-facing operations.

### Example of an Environment

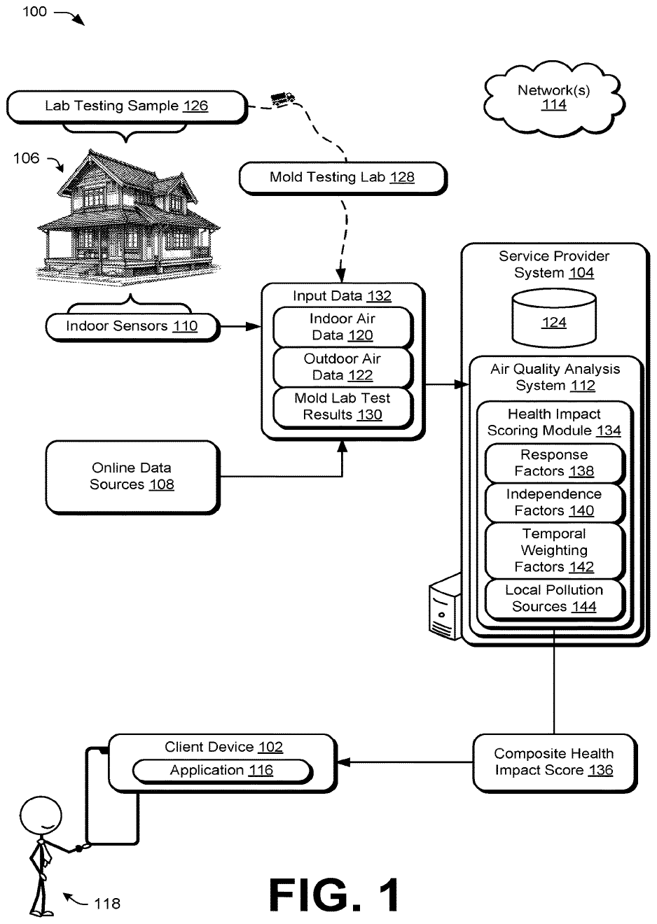

FIG. 1 is an illustration of an environment 100 in an example implementation that is operable to employ techniques described herein. The environment 100 includes a client device 102, a service provider system 104, a structure 106, and online data sources 108. As shown, the structure 106 is equipped with indoor sensors 110, and the service provider system 104 includes an air quality analysis system 112. In one or more implementations, the client device 102, the service provider system 104, the online data sources 108, the indoor sensors 110, and the air quality analysis system 112 are communicatively coupled, one to another, via network(s) 114. One example of the network(s) 114 is the Internet, although one or more of the client device 102, the service provider system 104, the online data sources 108, and the indoor sensors 110 may be communicatively coupled using one or more different connections or different networks in various implementations.

Although the air quality analysis system 112 is depicted in the environment 100 within the service provider system 104, an entirety or various portions of the air quality analysis system 112 are alternatively implemented at or by the client device 102 and/or the service provider system 104. In at least one implementation, for example, at least a portion of the air quality analysis system 112 is implemented by an application 116 of the client device 102 and/or using various resources of the client device 102, such as hardware resources, an operating system, firmware, and so forth. Alternatively or additionally, at least a portion of the air quality analysis system 112 is implemented by resources (e.g., server-based storage, processing, and so on) of the service provider system 104. Although not depicted, at least a portion of the air quality analysis system 112 is implemented using a third-party service in various examples, such as a web services platform that provides one or more hardware and/or other computing resources to support provision of services by web service providers.

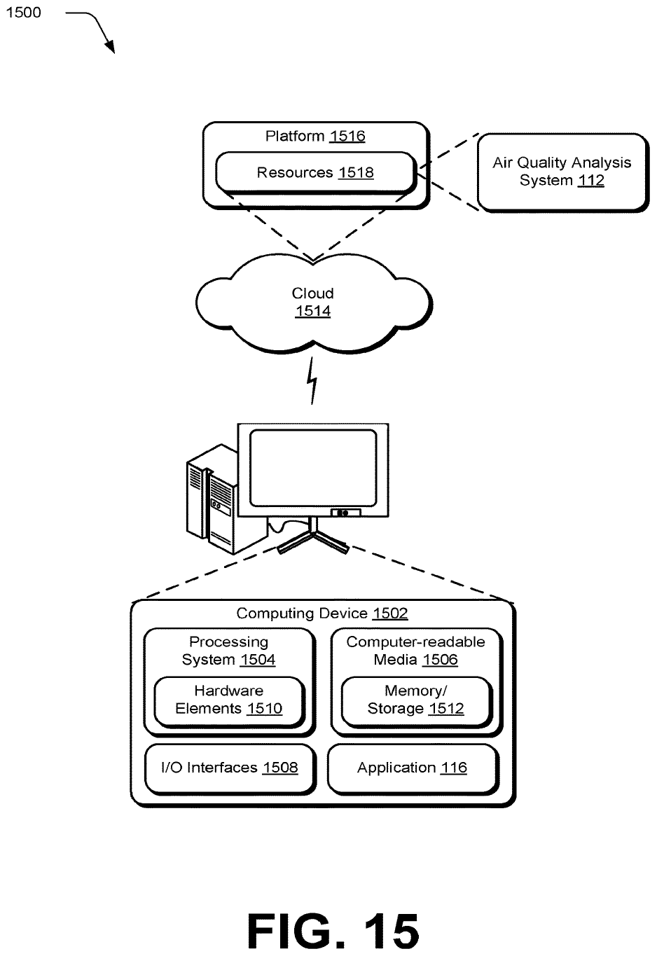

Computing devices that implement the environment 100 (e.g., the client device 102, the service provider system 104, and/or the third-party web services platform) are configurable in a variety of ways. A computing device, for instance, is configurable as a desktop computer, a laptop computer, a mobile device (e.g., assuming a handheld configuration such as a tablet or mobile phone), an IoT device, a wearable device (e.g., a smart watch, a ring, or smart glasses), an AR/VR device (e.g., the smart glasses), a server, and so forth. Thus, a computing device ranges from full resource devices with substantial memory and processor resources to low-resource devices with limited memory and/or processing resources. Additionally, although in instances in the following discussion reference is made to a computing device in the singular, a computing device is also representative of a plurality of different devices, such as multiple servers of a server farm or data center utilized to perform operations “over the cloud” as further described in relation to FIG. 15.

In at least one implementation, the application 116 supports communication of data across the network(s) 114, such as between the client device 102, the service provider system 104, and the air quality analysis system 112. By supporting such data communication, the application 116 provides a respective user of the client device 102 (and users of other client devices) access to digital services provided by the service provider system 104, such as the air quality analysis system 112. For example, the client device 102 receives data from the service provider system 104. Based on the received data, the application 116 causes various systems of the client device 102 to output user interfaces, such as by displaying user interfaces via display devices or making accessible voice-based user interfaces. For example, the client device 102 receives data from the service provider system 104 that includes composite health impact scores generated by the air quality analysis system 112, which may be output via user interfaces of the application 116 on the client device 102 to provide users with comprehensive scores that quantify a cumulative health impact from a plurality of pollutants.

In one or more implementations, users register with the service provider system 104 to obtain respective user accounts with the air quality analysis system 112. Such registration includes, for instance, an occupant 118 of the structure 106 (e.g., who is also a user of the client device 102) providing a physical address of the structure 106 to be analyzed for air quality and mold presence by the air quality analysis system 112. Additionally or alternatively, registration involves establishing a username and password combination for the user account. Subsequent to registering with the service provider system 104, computing devices (e.g., the client device 102) facilitate signing into, or otherwise authenticating to, the user account in various ways, such as by receiving a username and matching password, receiving biometric information (e.g., at least one image captured of a face or information captured of another body part such as a thumb or finger) that suitably matches stored biometric information associated with the user account, and so forth.

Although depicted as a house in the illustrated example, the structure 106 may be an office building, apartment complex, school, hospital, warehouse, factory, retail store, shopping mall, restaurant, hotel, gymnasium, theater, stadium, airport terminal, train station, greenhouse, barn, or any other type of enclosed or semi-enclosed space where air quality and mold presence monitoring may be beneficial. As shown, the structure 106 is equipped with various sensor systems to monitor environmental conditions inside the structure 106. Indoor sensors 110 comprise indoor climate sensors, indoor particulate matter (PM) sensors, and indoor gas sensors, which measure air data within the indoor environment of the structure 106. Although not illustrated, the structure 106 may include outdoor sensors in an outdoor environment at or near the structure. The outdoor sensors may be communicatively coupled to the client device 102, the service provider system 104, and the air quality analysis system 112 via the network(s) 114. The outdoor sensors may include outdoor climate sensors, outdoor particular matter (PM) sensors, and outdoor gas sensors, which measure air data in an outdoor environment near the structure. By way of example, the sensors (indoor and outdoor) are delivered to the structure 106 in response to the user registering a user account with and/or subscribing to an air quality and mold presence monitoring service offered by the service provider system 104 and/or the air quality analysis system 112.

In various examples, the climate sensors measure climate-related indicators in the air, including but not limited to, temperature, humidity, and pressure. These sensors contain physical transducers that convert environmental conditions into electrical signals. For instance, thermistors may be used to measure temperature changes, capacitive sensors to detect humidity variations, and piezoelectric elements to gauge pressure fluctuations. The electrical signals generated by these transducers are then digitized by analog-to-digital converters and transmitted to the air quality analysis system 112 for processing.

In yet other examples, the PM sensors measure particulate matter in the air, such as nano particles (less than 0.1 μm in diameter), PM1.0 particles (less than or equal to 1 μm in diameter), PM2.5 particles (less than 2.5 μm in diameter), and PM10 particles (less than or equal to 10 μm in diameter). These PM sensors may employ laser scattering technology, in which airborne particles pass through a laser beam, scattering light that is then detected by a photodiode. The intensity and pattern of scattered light are analyzed by onboard microprocessors to determine particle size and concentration. This data is then transmitted to the air quality analysis system 112 via digital communication protocols. Examples of pollutants within these particulate matter classes include dust, pollen, mold spores, smoke, smog, vehicle emissions, combustion byproducts, viruses, and so on.

The gas sensors measure various gases that may impact air quality, including but not limited to, volatile organic compounds (VOCs), carbon dioxide, formaldehyde, nitrogen dioxide, ozone, carbon monoxide, radon, methane, ethanol, propane, benzene, butane, hydrogen, hydrogen sulfide, and ammonia. These gas sensors may utilize electrochemical cells, metal oxide semiconductors, or infrared absorption techniques to detect specific gas molecules. The sensors generate electrical signals proportional to gas concentrations, which are then amplified, digitized, and transmitted to the air quality analysis system 112 for analysis. Individual climate parameters, individual gases, and individual particulate matter sizes may be referred to herein as air quality parameters. Moreover, the conglomeration of air quality parameters measured by the indoor sensors comprise indoor air data 120.

The air quality analysis system 112 may implement a sensor hardware integration framework to facilitate seamless data collection and processing from diverse sensor types. This framework may include a multi-protocol communication layer that supports various connectivity standards such as Wi-Fi, Bluetooth Low Energy, Zigbee, Z-Wave, and wired connections. The communication layer may employ a protocol translation framework to standardize data formats across heterogeneous sensors, implement automatic sensor discovery and configuration, and maintain persistent connections with failover mechanisms to ensure data continuity.

The sensor hardware integration framework may also include a sensor calibration and validation system. The system 112 may perform initial sensor calibration using reference measurements to establish baseline accuracy, conduct periodic recalibration through cross-sensor validation, e.g., by comparing readings across multiple sensors to identify and correct discrepancies. Drift detection algorithms may be implemented to identify gradual changes in sensor output over time, with compensation algorithms adjusting readings to maintain accuracy. The system 112 may apply environmental correction factors, adjusting sensor readings based on temperature and humidity conditions that can affect measurement accuracy. Additionally, the system 112 may employ anomaly detection techniques to automatically identify and flag sensor malfunctions, such as sudden spikes or drops in readings, or persistent deviations from expected values.

In some implementations, the framework may incorporate edge computing capabilities, which enables local pre-processing of sensor data, potentially reducing the amount of information transmitted, and conserving bandwidth. This may involve implementing sensor-specific noise reduction algorithms directly on the sensor hardware, filtering out irrelevant signals before data transmission. The system 112 may incorporate local data buffering mechanisms to store information during network connectivity issues, ensuring data continuity. Preliminary analysis for time-sensitive alerts may be executed locally, allowing for rapid response to critical conditions without relying on cloud processing. The edge implementation may optimize power usage through adaptive sampling techniques, adjusting data collection frequency based on detected environmental conditions or activity levels.

The sensor hardware integration framework may implement various hardware-specific data conditioning methods to standardize data across diverse sensor types. These methods may include applying sensor-specific correction coefficients obtained through laboratory calibration processes, adjusting raw sensor outputs to account for individual sensor characteristics and nonlinearities. Cross-platform normalization techniques may be employed to address manufacturer-specific biases, ensuring consistency in measurements across different sensor brands and models. For lower-quality sensors, the system 112 may utilize advanced signal processing techniques such as oversampling, noise filtering, or Kalman filtering to enhance measurement resolution and accuracy. Temporal alignment algorithms may be implemented to synchronize asynchronous data streams from multiple sensors, potentially using interpolation or resampling methods to create a unified time base for all sensor inputs.

The online data sources 108 refer to one or more online sources that provide various types of environmental, meteorological, and pollution-related data relevant to air quality analysis at the geographic location of the structure 106. Examples of online data sources 108 include government environmental monitoring agencies, meteorological services, air quality index (AQI) providers, traffic monitoring systems, industrial emission databases, satellite-based environmental monitoring platforms, and crowdsourced environmental data platforms. These sources may be publicly available (such as government databases) or private (such as commercial environmental monitoring services). The online data sources 108 collect and aggregate data on outdoor air quality parameters, meteorological conditions, traffic patterns, industrial emissions, local pollution sources, and other environmental factors that may impact air quality at specific geographical locations. In particular, outdoor air data 122 may be collected from the online data sources 108 and/or the outdoor sensors, which in some examples, represents an air quality history that includes concentrations of multiple pollutants and exposure patterns that illustrate how concentration levels at the geographic area vary over a duration, e.g., two years. In some implementations, the air quality analysis system 112 retrieves data from the online data sources 108 through application programming interfaces (APIs) that allow programmatic access to real-time and historical environmental information. By way of example, the air quality analysis system 112 communicates the physical address of the structure 106 to API endpoints of the online data sources 108, which return relevant environmental data associated with that geographic location or surrounding area.

In one or more implementations, the air quality analysis system 112 ingests indoor air data 120 and outdoor air data 122 through periodic data collection. At regular intervals, the indoor sensors 110 transmit packages containing their respective sensor data to the air quality analysis system 112. Additionally, the air quality analysis system 112 concurrently (e.g., at the same intervals) collects outdoor air data 122 from the outdoor sensors and/or requests a snapshot of the outdoor air data 122 at a given point in time. Upon receiving these data packages, the air quality analysis system 112 processes and stores the information as concurrent time series data. Notably, concurrent time series data refers to the simultaneous recording of multiple datasets (in this case, indoor air data 120 and outdoor air data 122) along a shared time axis. The service provider system 104 maintains the concurrent time series data in a storage device 124, which may be implemented as a database, file system, mass storage, virtual storage, or other data storage solution. In one or more implementations, for example, the storage device 124 may be virtualized across a plurality of data centers and/or cloud-based storage device(s). In this way, the outdoor air quality history may be requested directly from the online data source 108 and/or the outdoor air quality history may be retrieved from the storage device 124 at runtime.

This time-series approach allows for direct temporal comparisons between indoor and outdoor conditions, facilitating more comprehensive air quality analysis. Moreover, as the air quality analysis system 112 continues to collect data at regular intervals, the concurrent time series data in the data storage device 124 grows over time. This accumulation of data enables the system to perform trend analysis, identify patterns, and generate more accurate air quality assessments by analyzing both short-term fluctuations and long-term environmental changes affecting the structure 106.

As part of the air quality and mold presence monitoring service, the service provider system 104 facilitates periodic collection and analysis of lab testing samples 126 from the structure 106. When the occupant 118 registers for and/or subscribes to the service, the service provider system 104 may deliver sample collection materials to the structure 106. These materials typically include instructions and specialized equipment for collecting various types of samples that can be analyzed for the presence of mold and other pollutants. The lab testing sample 126 can take a variety of forms. Example sample types include, but are not limited to, surface samples obtained by swabbing or tape-lifting suspected moldy areas in the indoor environment, bulk samples collected by removing a portion of building material (e.g., drywall or carpet) within the indoor environment, air samples (e.g., collected from the indoor environment) using specialized air sampling devices. Additionally or alternatively, HVAC filters can be used as a sampling medium (e.g., by testing the HVAC filter itself or testing a swab rubbed on the HVAC filter), as HVAC filters naturally collect airborne particles within the indoor environment over an extended period.

Following the provided instructions, the occupant 118 collects the appropriate lab testing sample 126 and sends it to a designated mold testing lab 128, which conducts one or more tests on the sample. Examples of the mold lab tests include, but are not limited to, Polymerase Chain Reaction (PCR) testing to identify specific mold species through DNA analysis, culture testing to grow and identify viable mold spores, and direct microscopy to visually examine the sample for mold structures and other particulates. These tests not only detect the presence of mold but can also identify other airborne pollutants that impact indoor air quality.

In some scenarios, all three tests—PCR, culture testing, and direct microscopy—may be conducted on a single sample to provide a comprehensive analysis. PCR testing offers high sensitivity and specificity, allowing for precise identification of mold species even in small quantities. However, it may detect both viable and non-viable mold spores, potentially overestimating the current mold problem. Culture testing, on the other hand, identifies only viable mold spores, indicating active mold growth. This method may take several days to yield results and may not detect slow-growing or non-culturable mold species. Direct microscopy provides immediate results and can identify both mold spores and other particulates, but it may not distinguish between viable and non-viable spores or identify specific mold species. By utilizing all three tests in combination, the air quality analysis system 112 may gain a more complete understanding of the mold situation in the structure 106.

The air quality analysis system 112 receives the results of these lab tests, which may be stored in the storage device 124. As part of the air quality and mold presence monitoring service, this lab testing process may occur periodically, e.g., every three months. This accumulation of lab test results 130 over time allows the system to track changes in mold levels and other pollutants, correlating this information with the concurrent time series data of indoor and outdoor air data 120, 122. By integrating these diverse data sources, the air quality analysis system 112 can provide a more comprehensive and accurate assessment of indoor air quality and potential mold risks in the structure 106.

In accordance with the described techniques, one or more of the indoor air data 120, the outdoor air data 122, the mold lab test results 130, and other types of data from the online data sources 108 are packaged together as input data 132, which is input to the air quality analysis system 112 for analysis by a health impact scoring module 134. In various examples, the health impact scoring module 134 leverages deterministic algorithm(s) and/or machine learning model(s) to generate a composite health impact score 136 based on the input data 132. In implementations in which a machine learning model is leveraged, the machine learning model is trained and/or prompted to generate a composite health impact score 136 based on the input data 132. In some examples, the machine learning model is a publicly available pre-trained large language model (LLM) that is specifically prompted on how to analyze the input data 132. In other examples, the machine learning model is a fine-tuned variant of a publicly available LLM, refined on training data including a compendium of scientific studies that describe health impacts from various airborne pollutants and how the health impacts are impacted from combined exposure to different combinations of pollutants. In yet additional examples, the machine learning model is trained from scratch (e.g., starting with uninitialized or randomly initialized parameters) on the above-described training data.

Thus, in some implementations, the machine learning model is a proprietary model stored and executed on computing hardware (e.g., memory and processors) owned and operated by the service provider system 104. In implementations involving a publicly available model, however, the machine learning model is stored and executed on computing hardware owned and operated by a third-party service provider. In accordance with this approach, inputs and outputs exchanged between the air quality analysis system 112 and the machine learning model are facilitated via APIs. In implementations in which the health impact scoring module 134 implements deterministic algorithms, the service provider system 104 stores and runs the deterministic algorithm on proprietary computing hardware (e.g., memory and processors) owned and operated by the service provider system 104.

Although not depicted, the machine learning model, in some examples, is trained and/or prompted to generate a mold risk assessment based on the mold lab test results 130. The mold risk assessment focuses specifically on the likelihood of and health impact due to mold contamination within the indoor environment. In various example, the mold risk assessment includes a mold risk score, which quantifies the likelihood of (and/or health risk due to) active mold sources contaminating the air of the indoor environment. Importantly, the mold risk assessment and score concentrate on active mold sources that are currently releasing spores and mycotoxins into the air, rather than surface mold that may be years old and from an inactive source. This focus on active, airborne mold is crucial because it directly relates to human health impacts, as inhaled mold spores and associated compounds pose the greatest risk to occupants.

In at least one example, the laboratory mold tests are conducted on an HVAC filter previously installed at the structure 106, e.g., by performing PCR testing, culture testing, and/or microscopy testing on the HVAC filter itself. As part of this functionality, HVAC filters are periodically delivered to the structure 106 in accordance with their effective filtering lifespan. As part of the air quality and mold presence monitoring service, the occupant 118, after installing a newly delivered HVAC filter, initiates delivery of the old HVAC filter to the mold testing lab 128 for testing. Based on the replacement/delivery schedule of HVAC filters, the installation period of the tested HVAC filter is known and/or determinable by the air quality analysis system 112.

Here, the machine learning model is trained, prompted, and/or configured to estimate a total air volume processed by the HVAC filter based on the known installation period. As part of this, the machine learning model estimates HVAC runtime during the installation period based on (1) direct HVAC usage data (e.g. from a smart thermostat installed at the structure 106), (2) pressure changes in the indoor environment indicative of HVAC activation and deactivation, or (3) indoor-outdoor temperature differentials, e.g., an amount of HVAC usage estimated to maintain the differentials. Furthermore, the machine learning model is instructed to determine expected quantities of various mold species based on the processed air volume by the HVAC filter and a particulate matter capture rate associated with the HVAC filter, e.g., as determined from a minimum efficiency reporting value (MERV) rating for the HVAC filter. The machine learning model interprets the mold lab test results by comparing the expected quantities of the various mold species to observed quantities of mold species from the mold lab test results 130. In various examples, the mold risk assessment includes this comparative analysis of expected vs. observed mold quantities.

Returning to the discussion of the health impact scoring module 134, regardless of its technical implementation (e.g., deterministic algorithm(s) and/or machine learning model(s)), the health impact scoring module 134 is configured to generate a composite health impact score 136 based on the input data 132. Generally, the composite health impact score 136 represents a unified metric that quantifies the cumulative impact from exposure to multiple pollutants. Importantly, the composite health impact score 136 measures the cumulative effect of exposure to multiple pollutants simultaneously, rather than evaluating the impact of a single pollutant (associated with the highest exposure) in isolation.

Here, the input data 132 includes the air quality history of the outdoor air data 122 that includes concentrations of multiple pollutants at the geographic area and exposure patterns that illustrate how concentration levels at the geographic area vary over a duration, e.g., two years. In one or more implementations, the health impact scoring module 134 determines and applies response factors 138 to individual pollutants indicated by the input data 132. In some examples, the response factors 138 represent quantitative coefficients that translate pollutant concentrations into health impact metrics. Each response factor 138 may be specific to a particular pollutant and quantify the expected health impact per unit of exposure. The health impact scoring module 134 applies these response factors, for example, by multiplying a baseline concentration of each pollutant (e.g., the pollutant's average concentration over the relevant exposure duration) by its corresponding response factor to calculate an individual health impact score for that pollutant.

In various implementations, the health impact scoring module 134 determines and applies independence factors 140 to account for overlapping biological pathways and synergistic effects when multiple pollutants are present simultaneously. The independence factors 140 represent quantitative coefficients that adjust the cumulative health impact calculation to prevent overestimation that would occur if individual pollutant health impacts were simply added together. When multiple pollutants affect the same biological systems or pathways in the human body, their combined health impact may be less than the sum of their individual impacts due to shared mechanisms of action, saturation effects, or competitive interactions. In some examples, the health impact scoring module 134 applies these independence factors by multiplying the individual health impact scores (calculated using the response factors 138) by corresponding independence factor coefficients before combining them into the composite health impact score 136.

Additionally or alternatively, the health impact scoring module 134 determines and applies temporal weighting factors 142 to account for varying health impacts derived from different exposure patterns during the duration of exposure to the multiple pollutants. The temporal weighting factors 142 represent quantitative coefficients that adjust health impact calculations based on the timing, duration, and intensity patterns of pollutant exposure over time. These factors recognize that health impacts from pollutant exposure are not uniform across time. For instance, temporary spikes in pollutant concentrations may have disproportionately higher health impacts compared to sustained exposure at lower levels. The health impact scoring module 134 determines these temporal weighting factors 142 by analyzing the exposure patterns within the input data 132, identifying periods of elevated exposure, baseline exposure levels, temporary spikes in exposure, and exposure variability. Moreover, the health impact scoring module applies these temporal weighting factors 142 by multiplying the calculated health impact scores by corresponding temporal weighting coefficients that reflect the increased or decreased health significance of temporal exposure pattern characteristics.

In one or more implementations, the health impact scoring module 134 may incorporate local pollution sources 144 to enhance the accuracy of the composite health impact score 136 by accounting for proximity-based pollution exposure that may not be fully captured in regional air quality data. The local pollution sources 144 may include industrial facilities, manufacturing plants, power generation stations, waste treatment facilities, major roadways, airports, construction sites, agricultural operations, and other emission sources within a defined radius of the structure 106's geographic location. The local pollution sources 144 may be associated with emission metrics that quantify emissions of various pollutants from these local pollution sources 144. In one or more examples, the health impact scoring module 134 applies a local pollution modifier that modifies the composite health impact score 136 based on a quantity and proximity of the local pollution sources 144, as well as their associated emission metrics.

In various implementations, the composite health impact score 136 may quantify life expectancy loss attributable to cumulative pollutant exposure, providing a metric that translates complex air quality data into an understandable measure of potential longevity reduction. Additionally or alternatively, the composite health impact score 136 may quantify impacts on sub-clinical health outcomes that do not impact mortality. Examples of these sub-clinical health outcomes include mental clarity, morning congestion, productive capacity, hours of clear thinking per day, days of throat irritation per month, respiratory comfort, allergy symptoms, and the like. In various examples, the health impact scoring module 134 may determine a composite health impact score 136 for each of a plurality of sub-clinical health outcomes.

In accordance with the described techniques, the composite health impact score is measured relative to a pristine air baseline, which represents pre-industrialization exposure levels of various pollutants. The pristine air baseline may represent atmospheric conditions that existed before significant industrial activity, characterized by naturally occurring background concentrations of pollutants without anthropogenic contributions from sources such as fossil fuel combustion, industrial emissions, or vehicular traffic. Exemplary reference values for the pristine air baseline include but are not limited to: PM2.5: approximately 2.0 μg/m³ (range 1.5-3.0); Ozone: approximately 10-15 ppb; Nitrogen Dioxide: approximately 0.5-2.0 ppb; Sulfur Dioxide: approximately 0.1-0.5 ppb; and Carbon Monoxide: approximately 50-100 ppb. These ranges represent atmospheric conditions under which human physiology evolved. The system dynamically adjusts baseline values based on regional variations and emerging scientific evidence. Health impacts are calculated as the delta from these evolutionary baselines rather than from zero concentration or regulatory standards, providing a biologically-grounded assessment. That is, the response factors 138 translate pollutant concentrations into health impact metrics that are relative to the pristine air baseline.

For example, the composite health impact score 136 may measure life expectancy loss relative to the pristine air baseline, i.e., the mortality reduction based on the air that the occupant 118 breathes relative to pre-industrialization air. Additionally or alternatively, the composite health impact score 136 for a sub-clinical health outcome may measure a decrease in the sub-clinical health outcome relative to the pristine air baseline, i.e., the percentage decrease in mental clarity that the occupant 118 experiences based on the air that the occupant 118 actually breathes relative to pre-industrialization air.

It should be noted that the health impact scoring module 134 is employable to generate composite health impact scores 136 attributable to outdoor air at the geographic area, and composite health impact scores 136 attributable to indoor air in the structure. The input data 132 that serves as the basis for the outdoor computation includes concentrations of outdoor pollutants at the geographic area and exposure patterns that illustrate how concentration levels at the geographic area vary over a duration, e.g., two years. Similarly, the input data 132 that serves as the basis for the indoor computations includes concentrations of indoor pollutants at the structure 106 and exposure patterns that illustrate how concentration levels at the structure vary over a duration, e.g., two years. The concentrations of indoor pollutants may be measured by the indoor sensors 110, and extracted from the mold lab test results 130 and/or mold risk assessment. As explained above, the health impact scoring module 134 computes the composite health impact score 136 attributable to outdoor air by applying the response factors 138, independence factors 140, and temporal weighting factors 142 to the concentrations of the outdoor pollutants, and modifying the composite health impact score based on the local pollution sources 144.

To determine the composite health impact score attributable to the indoor air, the health impact scoring module 134 similarly applies the response factors 138, independence factors 140, and temporal weighting factors 142 to the concentrations of the indoor pollutants. The computation of the indoor composite health impact score 136 is context-aware of the outdoor composite health impact score 136 and/or outdoor air quality. That is, the health impact scoring module 134 accounts for the amount of outdoor air that penetrates into the indoor environment of the structure 106, estimating the infiltration rate based on building envelope and perforation data included in the input data 132. This building envelope and perforation data may include information about the structure's air tightness, ventilation systems, window and door sealing, construction materials, and structural integrity. Based on this estimated infiltration rate, the health impact scoring module 134 may attribute a corresponding portion of the outdoor composite health impact score 136 to the indoor composite health impact score 136, thereby accounting for the contribution of outdoor pollutants to the overall indoor air quality and health impact.

Having considered an example of an environment, consider now a discussion of some example details of the techniques for composite health impact scoring of air quality for multiple pollutants in accordance with one or more implementations.

### Composite Health Impact Scoring Implementation Details

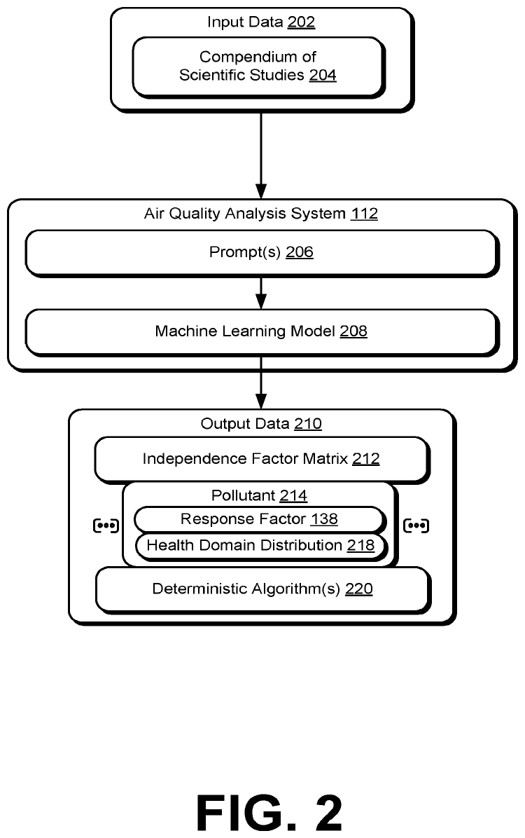

FIG. 2 depicts a system 200 in an example implementation showing operation of an air quality analysis system to leverage a machine learning model during a pre-processing phase for composite health impact scoring of air quality for multiple pollutants. As shown, the air quality analysis system 112 is illustrated as receiving input data 202 including a compendium of studies 204. The compendium of studies 204 comprises a comprehensive collection of peer-reviewed scientific research papers, epidemiological studies, toxicological assessments, and health impact analyses that document the relationships between pollutant exposure and human health outcomes. These studies include longitudinal cohort studies tracking health effects over extended periods, dose-response analyses quantifying health impacts at various exposure levels, meta-analyses synthesizing findings across multiple research efforts, and clinical studies examining acute and chronic health responses to specific pollutants. The compendium encompasses research on individual pollutants such as particulate matter, nitrogen dioxide, ozone, volatile organic compounds, and mold spores, as well as studies investigating synergistic effects and interactions between multiple pollutants. Additionally, the studies provide data on exposure-response relationships, biological pathways affected by different pollutants, vulnerable population responses, temporal patterns of health impacts, sub-clinical health outcomes including cognitive function, respiratory comfort, sleep quality, mental clarity, allergy symptoms, and productive capacity, and quantitative risk assessments that establish the scientific foundation for determining response factors and independence factors used in composite health impact scoring.

The air quality analysis system 112 is illustrated as providing one or more prompts 206 to a machine learning model 208. Generally, the machine learning model 208 is trained and/or prompted to generate output data 210 by analyzing, reasoning, and researching the compendium of scientific studies 204. The output data 210 provides the foundation for analyzing location-specific data during a runtime processing phase to determine the composite health impact score 136.

In some examples, the machine learning model 208 is a publicly available pre-trained large language model (LLM) that is specifically prompted on how to analyze the compendium of scientific studies 204. In other examples, the machine learning model 208 is a fine-tuned variant of a publicly available LLM, refined on task-specific data. In yet additional examples, the machine learning model 208 is trained from scratch (e.g., starting with uninitialized or randomly initialized parameters) on task-specific data. In some implementations, the machine learning model 208 is a proprietary model stored and executed on computing hardware (e.g., memory and processors) owned and operated by the service provider system 104. In implementations involving a publicly available model, however, the machine learning model 208 is stored and executed on computing hardware owned and operated by a third-party service provider. In accordance with this approach, inputs and outputs exchanged between the air quality analysis system 112 and the machine learning model 208 are facilitated via APIs.

The air quality analysis system 112 generates prompts 206 for provision to the machine learning model 208. Generally, the prompts 206 include the compendium of scientific studies 204 along with instructions on how to analyze the scientific literature to produce desired outputs. More specifically, the prompts 206 may include instructions specifying particular data points and/or concepts to analyze and/or focus on when making particular conclusions/outputs.

In one or more implementations, the machine learning model 208 processes the prompt(s) 206 to generate, as part of the output data 210, an independence factor matrix 212. The independence factor matrix 212 is a structured data representation that quantifies the degree of interaction and shared biological pathways between different pollutant combinations, providing coefficients (e.g., independence factors 140) that reduce overestimation when calculating cumulative health impacts from multiple simultaneous exposures. For example, the independence factor matrix 212 is a table containing the same set of pollutants on both axes. Here, an individual cell of the table contains an independence factor 140 between a pair of pollutants. For example, a cell in the carbon dioxide column and the sulfur dioxide row is an independence factor 140 (e.g., 0.7) quantifying the synergistic effect of combined exposure to carbon dioxide and sulfur dioxide. Notably, the cells in the independence factor matrix may correspond to a particular value of the independence factor 140 or a range of values for the independence factors.

To generate the independence factor matrix 212, the prompts 206 instruct the machine learning model 208 to focus on identifying synergistic and antagonistic effects between different combinations of pollutants, analyzing dose-response relationships for combined exposures, extracting information about shared mechanisms of toxicity, determining saturation effects in biological systems, evaluating competitive interactions between pollutants for the same biological targets, and quantifying the degree to which multiple pollutants affect overlapping physiological pathways such as respiratory inflammation, cardiovascular stress, and neurological function.

In various examples, an independence factor matrix 212 is generated to be leveraged for computing composite health impact scores 136 quantifying life expectancy loss. This independence factor matrix 212 may include independence factors 140 that calculate the synergistic effects of pollutant combinations on all-cause mortality. Additionally or alternatively, multiple independence factor matrices 212 are generated to be leveraged for computing composite health impact scores 136 quantifying sub-clinical health outcomes. A sub-clinical independence matrix 212 may include independence factors 140 that calculate the synergistic effects of pollutant combinations on a specific sub-clinical health outcome (e.g., mental clarity). Thus, the system 112 generates separate independence factor matrices 212 for all-cause mortality, and various sub-clinical health outcomes.

In an illustrative example, the cell corresponding to the pollutant combination of ozone with PM2.5 may be a range (0.2 to 0.4) or a particular value (0.3) in the all-cause mortality matrix 212. The cell corresponding to the same combination of ozone with PM2.5 may be a different range (0.4-0.7) or particular value (0.55) in a sub-clinical matrix 212, e.g., quantifying independence factors 140 for mental clarity. In another illustrative example, the cell corresponding to the pollutant combination of nitrogen dioxide with PM2.5 may be a range (0.15-0.35) or a particular value (0.25). The cell corresponding to the same combination of nitrogen dioxide with PM2.5 may be a different range (0.25-0.45) or particular value (0.35) in a sub-clinical matrix 212, e.g., quantifying independence factors 140 for mental clarity.

In one or more implementations, the machine learning model 208 considers particle characterization data to refine the independence factors 140. The particle characterization data comprises detailed information about particle composition, emission source attribution, and chemical fingerprints that distinguish between different types of particles beyond mass measurements. By incorporating particle characterization to refine the independence factors 140, the system recognizes that combustion-derived particles share more biological pathways than secondary inorganic aerosols, requiring different independence factor adjustments. For example, diesel exhaust particles and biomass burning particles may exhibit greater pathway overlap and thus require lower independence factors when present together, compared to combinations involving secondary sulfate or nitrate particles that affect different biological mechanisms.

In one or more implementations, the machine learning model 208 processes the prompt(s) 206 to generate, as part of the output data 210, a response factor 138 for a plurality of pollutants 214. As previously mentioned, the response factor 138 for a particular pollutant 214 is a quantitative coefficient that translates pollutant concentrations into a health impact metric. The response factor 138 for a pollutant 214 is designed to be multiplied with a concentration of the pollutant 214 to quantify a health impact from exposure to that pollutant 214 at the concentration. To generate the response factors 138, the prompts 206 instruct the machine learning model 208 to focus on extracting dose-response relationships from epidemiological studies, identifying threshold concentrations where health effects begin to manifest, and quantifying the magnitude of health impacts per unit of pollutant exposure across different population groups and exposure durations. The machine learning model 208 may also analyze studies that establish baseline health metrics, mortality rates, and sub-clinical health outcomes to calibrate the response factors against measurable health endpoints.

In one or more implementations, the machine learning model 208 processes the prompt(s) 206 to generate, as part of the output data 210, a health domain distribution 218 for each of the pollutants 214. The health domain distribution 218 represents a quantitative breakdown of how a particular pollutant 214's health impacts are distributed (e.g., percentage-wise) across different physiological systems and health domains, such as respiratory function, metabolic function, cardiovascular health, neurological effects, immune system response, cancer risk, reproductive health, and dermatological impacts. To generate the health domain distribution 218, the prompts 206 instruct the machine learning model 208 to focus on identifying which organ systems and biological pathways are primarily affected by each pollutant 214, quantifying the relative severity of impacts across different health domains, analyzing dose-response relationships specific to each health domain, extracting information about acute versus chronic effects within each physiological system, and determining the percentage allocation of total health impact attributable to each affected health domain for comprehensive risk assessment.

In one or more implementations, the machine learning model 208 processes the prompt(s) 206 to generate, as part of the output data 210, one or more deterministic algorithms 220. The deterministic algorithms 220 comprise computational procedures that systematically and algorithmically combine pollutant concentrations and exposure patterns with the response factors 138, independence factors 140, temporal weighting factors 142, multipliers from local pollution sources 144, atmospheric amplification factors, and other parameters to calculate the composite health impact score 136 in a consistent and reproducible manner. These algorithms define the mathematical operations, weighting schemes, and computation sequences used to transform location-specific input data (e.g., pollutant concentration data, pollutant exposure patterns, local pollution sources, and terrain and topographical data) into quantified health impact metrics, providing a standardized framework for processing location-specific air quality data during runtime operations without utilizing additional machine learning inference.

That is, in various implementations, the air quality analysis system 112 leverage the machine learning model 208 to analyze, research, and reason about the large amounts of input data 202 in the compendium of scientific studies 204 during a pre-processing stage to develop a deterministic algorithm 220 for runtime processing. At runtime, the deterministic algorithm 220 leverages inputs describing location-specific air quality data to determine the composite health impact score 136. This two-stage processing approach significantly improves computer performance and resource utilization by separating computationally intensive machine learning operations from time-sensitive runtime calculations. During the pre-processing stage, the system can leverage high-performance computing resources to thoroughly analyze the scientific literature without time constraints, generating optimized deterministic algorithms that encode the complex relationships between pollutants and health impacts. At runtime, the system achieves faster response times and reduced computational overhead by executing the pre-computed deterministic algorithms rather than performing resource-intensive machine learning inference, thereby enabling real-time or near-real-time composite health impact scoring while minimizing processing latency, memory usage, and energy consumption during user-facing operations.

In one or more implementations, the air quality analysis system 112 includes and/or leverages a set of mortality-specific prompt(s) 206 for generating the output data 210 that provides the foundation for generating the composite health impact score 136 that quantifies life expectancy loss. Additionally or alternatively, the air quality analysis system 112 includes and/or leverages multiple sets sub-clinical-specific prompt(s) 206, where each set is specific to a particular sub-clinical health outcome. A sub-clinical-specific set of prompt(s) 206 for a particular sub-clinical health outcome (e.g., mental clarity) include instructions for generating the output data 210 that provides the foundation for generating the composite health impact score that quantifies the impact to that particular sub-clinical health outcome (e.g., mental clarity). As a result, a deterministic algorithm 220 is output for determining a composite health impact score 136 quantifying life expectancy loss, and multiple deterministic algorithms 220 are output for determining composite health impact scores 136 quantifying impact on sub-clinical health outcomes.

In various implementations, the machine learning model 208 may re-process the compendium of studies 204 in response to certain trigger events that affect the relevance or completeness of the scientific literature knowledge base. For example, when the compendium of studies 204 is modified, such as when a previously included study is determined to be irrelevant to air quality health impacts and is subsequently removed from the compendium, or when new peer-reviewed research is published that provides updated findings on pollutant health effects, synergistic interactions, or exposure-response relationships. In such cases, the air quality analysis system 112 may initiate a re-processing cycle where the machine learning model 208 analyzes the updated compendium of studies 204 to generate revised output data 210, including updated independence factor matrices 212, response factors 138, health domain distributions 218, and deterministic algorithms 220. This adaptive approach ensures that the composite health impact scoring methodology remains current with the latest scientific understanding and maintains accuracy as new research emerges or existing studies are re-evaluated for their applicability to air quality health assessment.

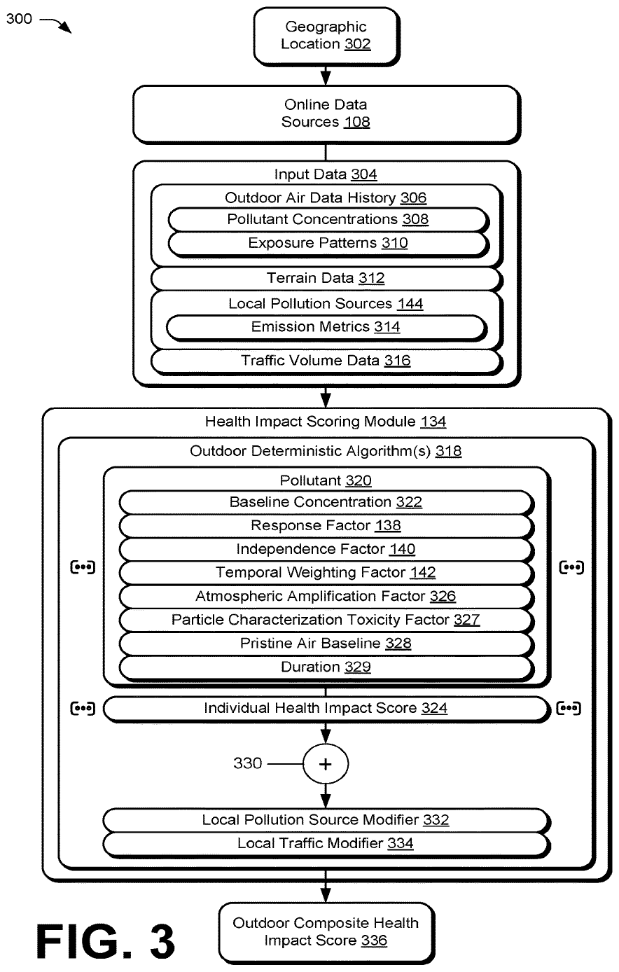

FIG. 3 depicts a system 300 in an example implementation showing operation of a health impact scoring module to leverage a deterministic algorithm during a runtime processing phase for composite health impact scoring of outdoor air quality for multiple pollutants. In the system 300, the online data sources 108 are illustrated as receiving a geographic location 302. For example, the client device 102 transmits the geographic location 302 to the service provider system 104 via an API call, which in turn queries the online data sources 108 through their respective APIs using the geographic location 302 as a parameter, thereby triggering the online data sources 108 to provide relevant environmental and air quality data associated with that geographic location 302 back to the service provider system 104.

The online data sources 108 are illustrated as providing a variety of input data 304 to the service provider system 104, e.g., including the health impact scoring module 134. The input data 304 includes an outdoor air data history 306. The outdoor air data history 306 may be retrieved from the online data source 108. Additionally or alternatively, the outdoor air data history 306 may be collected periodically by the outdoor sensors, stored in the storage device 124, and then retrieved from the storage device 124.

As shown, the outdoor air data history 306 includes pollutant concentrations 308 and exposure patterns 310. The outdoor air data history 306 represents a comprehensive record of environmental conditions at the geographic location over an extended time period, e.g., spanning multiple years to capture seasonal variations and long-term trends. The pollutant concentrations 308 comprise measured levels of various airborne contaminants including particulate matter (PM2.5, PM10), nitrogen dioxide, ozone, sulfur dioxide, carbon monoxide, volatile organic compounds, and other pollutants that impact air quality at the specific geographic location 302. The exposure patterns 310 illustrate temporal variations in pollutant levels, showing how concentrations fluctuate throughout different time periods such as daily cycles, seasonal changes, weather-related variations, episodic pollution events, and temporary concentration spikes. Additionally or alternatively, the outdoor air data history 306 includes air quality events associated with the geographic location 302 during the period of evaluation, such as wildfires, dust storms, industrial accidents, or other events that impact air quality temporarily or permanently.

The outdoor air data history 306 may be represented as time-series data comprising data points corresponding to specific temporal measurements of pollutant concentrations 308, thus capturing both the pollutant concentrations 308 and the exposure patterns 310. In some implementations, the outdoor air data history 306 may be structured as a graph illustrating concentration levels at various points in time during the duration of evaluation. These points in time can be at any granularity such as daily, hourly, minutely, or even more frequent intervals depending on the monitoring capabilities and data availability from the online data sources 108. Examples of online data sources 108 that may provide the outdoor air data history 306 (or portions thereof) include government environmental monitoring agencies, meteorological services, community-based air quality monitoring networks, satellite-based environmental platforms, state and local air quality management districts, industrial emission reporting databases, and commercial environmental data providers that aggregate information from multiple monitoring stations and sensors.

In one or more implementations, the input data 304 includes terrain data 312. The terrain data 312 comprises geographical and topographical information about the physical landscape surrounding the geographic location 302, including elevation profiles, slope gradients, valley configurations, mountain ranges, coastal features, urban canyon effects, and natural barriers that influence air flow patterns and pollutant dispersion. This terrain data 312 is crucial for understanding how local topography affects pollutant concentration and distribution, as features such as valleys can trap pollutants while elevated areas may experience different wind patterns and air circulation. Examples of online data sources 108 that provide terrain data 312 include government geological survey databases, satellite-based digital elevation models, national oceanic and atmospheric administration databases, geospatial data platforms, high-resolution topographic data repositories, and commercial geographic information system providers that offer detailed topographical mapping and elevation data for air quality modeling applications.

In one or more implementations, the input data 304 includes the local pollution sources 144. In some examples, the service provider system 104 may maintain a local pollution source database (e.g., in the storage device 124) containing a curated list of establishment types and their associated emission metrics 314. Examples of establishment types include manufacturing facilities, chemical plants, power generation stations, refineries, waste treatment facilities, airports, gas stations, golf courses, construction sites, printing shops, parking lots, agricultural operations, and commercial food processing facilities.

The emission metrics 314 comprise quantitative data that characterizes the pollutant output from various sources, including both numerical measurements and qualitative assessments of emission levels. Examples of emission metrics 314 include emission rates of various pollutants, e.g., measured in units such as tons per year or kilograms per day. Additionally, the emission metrics 314 may include composite scores quantifying predicted overall pollutant emission, as well as qualitative descriptors such as low, medium, or high predicted emission rate classifications for individual pollutants.

The service provider system 104 may query one or more online data sources 108 for a list of establishments within a specified radius of the geographic location 302. Examples of online data sources 108 that may provide this information such as business directory services, government facility databases, commercial mapping platforms, or regulatory compliance databases. In response to receiving this list of establishments, service provider system 104 may cross-reference the obtained establishments against the local pollution source database to identify which establishments qualify as local pollution sources 144 and retrieve their corresponding emission metrics 314 for incorporation into the composite health impact score calculation.

Additionally or alternatively, the local pollution sources 144 include registered emitters, which are facilities that are officially documented and monitored by environmental regulatory agencies for their pollutant emissions. The registered emitters are retrieved from online data sources 108 in the form of regulatory databases such as the Environmental Protection Agency's (EPA) Toxics Release Inventory (TRI), National Emissions Inventory (NEI), and Greenhouse Gas Reporting Program (GHGRP), EPA's Enforcement and Compliance History Online (ECHO) database, state environmental agency databases, and regional air quality management district reporting systems. These registered emitters are associated with emission metrics that may include more direct and facility-specific data in comparison to the generalized emission metrics based on establishment categories. For instance, while database-based emission metrics may consist of score metrics and qualifiers that generalize certain types of establishments and treat each establishment of that type uniformly, registered emitters may be associated with more precise data such as actual measured emission rates of various pollutants (e.g., reported in tons per year) of individual facilities.

In one or more implementations, the input data 304 includes traffic volume data 316. The traffic volume data 316 comprises quantitative measurements of vehicular activity within a specified radius of the geographic location 302, including identification of major roadways passing within the radius of the geographic location 302 and traffic volume statistics for each roadway, daily traffic counts, vehicle classification data distinguishing between passenger cars, trucks, buses, and motorcycles, and average daily traffic patterns. This traffic volume data 316 is significant for air quality analysis because vehicular emissions constitute a major source of pollutants including nitrogen oxides, particulate matter, carbon monoxide, and volatile organic compounds that directly impact local air quality conditions. Examples of online data sources 108 that may provide traffic volume data 316 include government transportation departments that maintain traffic monitoring systems, municipal traffic management agencies that operate automated counting stations, highway administration databases that track vehicle flow on major roadways, urban planning departments that collect transportation data for city planning purposes, and commercial traffic analytics platforms that aggregate vehicular movement data from various monitoring technologies such as loop detectors, cameras, and mobile device tracking system.

Generally, the health impact scoring module 134 is configured to process the input data 304 using one or more outdoor deterministic algorithms 318, e.g., of the deterministic algorithms 220 output by the machine learning model 208 during the pre-processing stage. To do so, the outdoor deterministic algorithm(s) 318 analyze a plurality of pollutants 320 indicated by the outdoor air data history 306 as present at the geographic location 302. In particular, the outdoor deterministic algorithm(s) 318 generate individual health impact scores 324 attributable to individual pollutants 320, and then aggregate the individual scores to generate the outdoor composite health impact score 336. In the following discussion, operation of the outdoor deterministic algorithms(s) 318 is discussed in the context of an individual pollutant 320, but this process is repeated for each pollutant 320 detected at the geographic location 302.

Given a pollutant 320, for instance, the health impact scoring module 134 extracts a baseline concentration 322 of the pollutant 320 from the outdoor air data history 306. The baseline concentration 322 may be an average concentration of the pollutant 320 over the relevant duration of evaluation. In some examples, the baseline concentration 322 may exclude outliers, such as extreme concentration values that deviate significantly from typical levels, to provide a more representative measure of typical exposure conditions.

In accordance with the described techniques, the outdoor deterministic algorithm(s) 318 apply the response factor 138 of the pollutant 320. As previously described, the response factor 138 is a quantitative coefficient that translates pollutant concentrations into health impact metrics. The health impact scoring module 134 applies the response factor 138 by multiplying the baseline concentration 322 of the pollutant 320 by the corresponding response factor 138 to calculate an individual health impact score 324 for that pollutant 320.

Moreover, the outdoor deterministic algorithm(s) 318 apply the independence factor 140 specific to the combination of pollutants 320 detected at the geographic location 302. The independence factors 140 are extracted from the independence factor matrix 212 generated during the pre-processing stage. As previously mentioned, the independence factor matrix 212 is structured as a table containing the same set of pollutants on both axes, where individual cells contain independence factors quantifying the synergistic effects between specific pollutant combinations. To apply these independence factors, the outdoor deterministic algorithm(s) 318 first identify a primary contributor among the detected pollutants 320, which is typically the pollutant with the highest concentration relative to its pristine air baseline. Secondary contributors are the remaining pollutants that contribute to the overall health impact but at lower relative concentrations compared to the primary contributor.

For example, when evaluating two pollutants such as nitrogen dioxide (NO₂) and ozone, the system may identify NO₂ as the primary contributor due to its higher relative concentration at the geographic location 302. The primary contributor's independence factor is assigned a value of 1.0, meaning its full health impact is retained without reduction. For the secondary contributor (ozone), the independence factor is extracted from the independence factor matrix 212 by locating the cell corresponding to the row and column combination of ozone and NO₂. If this cell contains an independence factor of 0.7, the health impact score calculated for ozone would be multiplied by 0.7 to account for the overlapping biological pathways and reduced cumulative effect when both pollutants are present simultaneously.

When three or more pollutants 320 are present, such as NO₂ (primary contributor), ozone, and PM2.5 (both secondary contributors), the independence factors 140 for the secondary contributors are determined by applying a weighting function based on their concentrations relative to their respective pollutant-specific pristine air baselines. As previously mentioned, the pristine air baseline for a pollutant 320 represents the naturally occurring background concentration of the pollutant 320 before significant industrial activity. For instance, if ozone has a baseline concentration of 60 μg/m³ above its pristine air baseline and PM2.5 has a baseline concentration of 30 μg/m³ above its pristine air baseline, their respective weights would be 0.67 (60/90) and 0.33 (30/90). The independence factors 140 extracted from the matrix 212 for ozone-NO₂ (0.7) and PM2.5-NO₂ (0.8) combinations would then be weighted accordingly: ozone independence factor=0.7×0.67=0.47, and PM2.5 independence factor=0.8×0.33=0.26. A pollutant 320's weighted independence factor 140 is then applied to its respective individual health impact score 324.

In various implementations, the outdoor deterministic algorithm(s) 318 apply the temporal weighting factor 142 derived from the pollutant 320's exposure patterns 310. As previously mentioned, the temporal weighting factor 142 represents a quantitative coefficient that adjusts health impact calculations based on the timing, duration, and intensity patterns of pollutant exposure over time. To determine the temporal weighting factor 142, for example, the outdoor deterministic algorithm (408s) 318 analyze the exposure patterns 310 by calculating the variance in pollutant concentrations over the evaluation period, identifying the frequency and magnitude of concentration spikes above baseline levels, determining the duration of elevated exposure periods, and computing a weighted average that assigns higher coefficients to periods with greater concentration variability and more frequent exposure spikes. The temporal weighting factor 142 is applied by multiplying the individual health impact score 324 by the temporal weighting coefficient. For example, if a pollutant has a baseline concentration 322 that results in an individual health impact score of 10, but the exposure pattern shows frequent concentration spikes that are twice the baseline level occurring 20% of the time, the temporal weighting factor might be calculated as 1.3, resulting in an adjusted health impact score of 13 (10×1.3) to account for the increased health significance of the variable exposure pattern.

Additionally or alternatively, the outdoor deterministic algorithm(s) 318 apply an atmospheric amplification factor 326 derived from the terrain data 312 at the geographic location 302. The atmospheric amplification factor 326 represents a quantitative coefficient that adjusts pollutant concentration measurements to account for local meteorological and topographical conditions that influence air flow patterns, pollutant dispersion, and accumulation effects at the specific geographic location 302. The outdoor deterministic algorithm(s) 318 determine the atmospheric amplification factor 326 by analyzing terrain data 312 including elevation profiles, valley configurations, coastal proximity, urban canyon effects, prevailing winds (including direction and magnitude) and natural barriers, then calculating how these topographical features impact air circulation and pollutant retention at the geographic location 302. The atmospheric amplification factor 326 is applied by multiplying the individual health impact score 324 by the amplification coefficient, thereby adjusting the health impact calculations to reflect the actual pollutant exposure conditions influenced by local geography.

For example, in a valley location such as the San Fernando Valley in California, surrounding mountain ranges may create a natural bowl that traps pollutants during temperature inversion events, leading to an atmospheric amplification factor greater than 1.0 that increases the effective concentration of all detected pollutants. Conversely, at a coastal location such as Santa Monica, California, consistent ocean breezes may facilitate pollutant dispersion and dilution, resulting in an atmospheric amplification factor less than 1.0 that reduces the effective concentration of pollutants compared to their measured baseline levels. In various implementations, the atmospheric amplification factor 326 may be applied uniformly across all pollutants 320 detected at the geographic location 302, as the topographical and meteorological conditions that influence air circulation patterns generally affect pollutant dispersion in a similar manner regardless of the specific pollutant type.

In one or more implementations, the outdoor deterministic algorithm(s) 318 apply a particle characterization toxicity factor 327 to the individual health impact score 324 of particulate matter pollutants 320. The particle characterization toxicity factor 327 represents a quantitative coefficient that adjusts health impact calculations based on the composition and source characteristics of particles, recognizing that particles of identical mass can exhibit vastly different biological responses depending on their chemical composition and origin. This toxicity weighting factor accounts for the 10-100 fold variation in particle toxicity that mass-only measurements cannot distinguish. Exemplary weighting ranges include: diesel exhaust particles exhibiting 5.0-10.0 relative toxicity; biomass burning particles showing 3.0-7.0 relative toxicity; traffic emissions demonstrating 4.0-8.0 relative toxicity; coal combustion particles having 2.0-5.0 relative toxicity; secondary sulfate particles showing 0.5-1.5 relative toxicity; secondary nitrate particles exhibiting 1.0-2.0 relative toxicity; and sea salt aerosol particles demonstrating 0.1-0.5 relative toxicity. Notably, the baseline with reference to which these relative toxicities are measured may correspond to pristine air particulate matter, e.g., the average toxicity of particulates from sources under pre-industrial atmospheric conditions.

The outdoor deterministic algorithm(s) 318 may determine the particle characterization toxicity factor 327 by analyzing various types of data available from the online data sources 108 that enable source attribution via characteristic chemical fingerprints. Examples of this data include meteorological conditions that indicate atmospheric processing pathways, emission inventory databases that identify predominant pollution sources within the geographic area, satellite-based aerosol optical depth measurements that provide information about particle light-scattering properties, air quality monitoring station data that includes speciated particulate matter measurements distinguishing characteristic chemical fingerprints of particulate matter (organic carbon, elemental carbon, sulfate, nitrate, hopanes, levoglucosan, potassium, endotoxins, fungal markers), wildfire and prescribed burn databases that indicate periods of biomass burning influence, traffic density and fleet composition data that suggests the prevalence of diesel versus gasoline emissions, industrial facility emissions reporting that identifies coal combustion or other specific source contributions, and seasonal patterns in pollutant ratios that may indicate secondary aerosol formation versus primary emissions.

By correlating these data streams, the algorithm 318 may estimate the likely composition profile of particulate matter at the geographic location 302 through source attribution via characterization. This approach may identify particle origins through characteristic chemical fingerprints, enabling the system to distinguish between various emission sources including traffic emissions (characterized by hopanes and elemental carbon), biomass burning (identified by levoglucosan and potassium), secondary aerosols (marked by sulfate and nitrate), and biogenic particles (distinguished by endotoxins and fungal markers). The algorithm 318 applies statistical models that associate these specific source signatures with established toxicity weightings. For example, the algorithm 318 may assign higher toxicity factors during periods when meteorological conditions and emission patterns suggest predominant diesel exhaust or biomass burning contributions, while applying lower factors when data indicates secondary sulfate or sea salt aerosol dominance.

Additionally or alternatively, the outdoor deterministic algorithms leverage input data 304 from the outdoor sensors and laboratory testing to determine the particle characterization toxicity factor 327. Laboratory sample collection for outdoor air particle characterization may be conducted using specialized sampling equipment deployed at or near the geographic location 302, including high-volume air samplers that collect particles on filters for subsequent chemical analysis, cascade impactors that separate particles by size for size-specific composition analysis, and active sampling devices that draw ambient air through collection media over predetermined time periods (e.g., 30-day integrated samples). The collected samples are then analyzed using techniques such as chemical speciation via X-ray fluorescence spectroscopy for elemental composition, ion chromatography for ionic species identification, thermal-optical analysis for organic and elemental carbon fractionation, gas chromatography-mass spectrometry for organic molecular markers, biological characterization for endotoxins, allergens, and mycotoxins, as well as oxidential potential assays measuring capacity to generate reactive oxygen species.

In one or more implementations, outdoor sensors may be deployed at or near the geographic location 302 to provide continuous particle characterization data that facilitates determination of the particle characterization toxicity factor 327. These outdoor sensors may include multi-wavelength optical analyzers that measure particle size distributions across multiple size ranges, enabling differentiation between fine and coarse particulate matter fractions that exhibit different toxicity profiles. The sensors may employ laser-based light scattering and absorption measurements at multiple wavelengths to determine optical properties including scattering coefficients and absorption coefficients, which serve as indicators of particle composition and source characteristics. Through multi-wavelength analysis, the sensors may provide preliminary composition indicators that enable detection of combustion particles versus secondary aerosols based on their distinct optical signatures, where combustion-derived particles typically exhibit higher absorption coefficients due to their elemental carbon content, while secondary aerosols show predominantly scattering behavior. The continuous sensor data may be processed using algorithms that correlate optical properties with known particle types, enabling the system to estimate toxicity weighting factors based on the predominant particle characteristics detected at each measurement interval.

In one or more implementations, the system 112 may implement a temporal fusion algorithm that combines the continuous sensor data with periodic laboratory results using the machine learning model 208 to estimate time-resolved particle composition between sampling intervals, thereby maintaining characterization continuity while controlling analytical costs. That is, the temporal fusion algorithm correlates the sensor measurements with the lab results to enhance accuracy in particle characterization, chemical composition analysis, and emission source attribution. The temporal fusion algorithm enables source attribution via characterization by identifying particle origins through characteristic chemical fingerprints, distinguishing traffic emissions (characterized by hopanes and elemental carbon), biomass burning (identified by levoglucosan and potassium), secondary aerosols (marked by sulfate and nitrate), and biogenic particles (distinguished by endotoxins and fungal markers). By correlating these continuous sensor measurements with periodic laboratory analysis, the algorithm generates the particle characterization toxicity factors 327 that reflect the varying composition of particulate matter, enabling the system to apply composition-specific toxicity weightings to various particulate matter pollutants 320.

In some examples, the outdoor deterministic algorithm(s) 318 consider particle characterization data when determining the atmospheric amplification factor 326. As discussed above, the particle characterization data comprises detailed information about particle composition, emission source attribution, and chemical fingerprints that distinguish between different types of particles beyond simple mass measurements. The atmospheric amplification factor incorporates particle characterization by recognizing that meteorological conditions differentially affect particles based on their composition and hygroscopic properties. Component ranges for amplification calculation include: (1) Temperature Inversion Effect, e.g., recognizing stronger effects on combustion particles versus sea salt; (2) Wildfire Smoke Aging, e.g., recognizing atmospheric processing increases toxicity of organic aerosols; (3) Urban Canyon Trapping, e.g., recognizing that Urban Canyon Trapping primarily affecting traffic emissions; (4) Humidity Effects based on particle hygroscopicity; and (5) Photochemical Processing for secondary organic aerosol formation. The system adjusts amplification based on characterized particle types, recognizing that hygroscopic particles (sulfate, nitrate) behave differently than hydrophobic particles (fresh soot, diesel exhaust) under varying meteorological conditions.

Notably, a pollutant 320's individual health impact score 324 is computed relative to its pristine air baseline 328. As previously discussed, the pollutant 320's pristine air baseline 328 represents pre-industrialization atmospheric concentrations of the pollutant 320. To compute the individual health impact score 324 relative to this baseline, the outdoor deterministic algorithm(s) 318 may determine the baseline concentration 322 as the amount or proportion above the pristine air baseline 328, rather than as an absolute concentration value. For example, if a pollutant has a measured concentration of 50 μg/m³ and its pristine air baseline is 10 μg/m³, the baseline concentration 322 used in calculations would be 40 μg/m³ (the excess above pristine conditions). Additionally or alternatively, when determining weighting coefficients for independence factors 140 in multi-pollutant scenarios, the system grounds these calculations in pristine air baseline quantities by comparing each pollutant 320's concentration relative to its respective pristine air baseline.

In one or more implementations, the outdoor deterministic algorithm(s) 318 apply a duration 329 to a pollutant 320's individual health impact score 324. The duration 329 represents the time period over which exposure to the pollutant 320 is evaluated, which may range from short-term acute exposure periods (such as hours or days) to long-term chronic exposure assessments spanning years or decades. The duration 329 is applied by scaling the individual health impact score 324 based on the length of exposure, recognizing that health impacts from pollutant exposure are cumulative and that longer exposure periods generally result in greater overall health consequences. In some cases, the outdoor deterministic algorithm(s) 318 extrapolate exposure over a lifetime by projecting current pollutant concentration levels and exposure patterns forward to estimate the total cumulative health impact an individual would experience if they remained at the geographic location 302 for their entire lifespan. For example, if a pollutant 320 has an individual health impact score 324 of 0.5 health impact units per year of exposure, and the duration 329 is set to evaluate lifetime exposure of 75 years, the algorithm would multiply 0.5 by 75 to generate a total individual health impact score of 37.5 health impact units, representing the cumulative health consequence of lifelong exposure to that pollutant at the current concentration levels.

As shown at 330, the individual health impact scores 324 for the pollutants 320 detected at the geographic location 302 are aggregated. Furthermore, a local pollution source modifier 332 and a local traffic modifier 334 are applied to the aggregation result to account for the local pollution sources 144 and vehicle traffic near the geographic location 302.

The local pollution source modifier 332 is a quantitative adjustment factor that accounts for the additional health impact from pollution sources located within a specified radius of the geographic location 302. The modifier 332 is determined algorithmically by identifying all local pollution sources 144 within the radius, retrieving their associated emission metrics 314 from the local pollution source database, calculating distance-weighted contributions based on proximity to the geographic location 302, aggregating these contributions into a composite modifier coefficient that reflects the cumulative impact of nearby emission sources. The local pollution source modifier 332 is applied by multiplying the aggregated individual health impact scores 324 by the modifier coefficient, thereby increasing the composite health impact score 136 to account for localized pollution exposure that may not be fully reflected in broader regional air quality measurements.

For example, if a geographic location 302 is situated within 2 miles of a chemical manufacturing plant, a power generation station, and a major highway interchange, the algorithm would retrieve emission metrics for each source, apply distance-based weighting factors (with closer sources receiving higher weights), and generate a local pollution source modifier of 1.4 to reflect the additional health risk from these proximate pollution sources. While the local pollution source modifier 332 is depicted and described above as being applied to the aggregated individual health impact scores 324 from multiple pollutants 320, the local pollution source modifier 332 may alternatively be computed and applied on a per-pollutant basis, where each pollutant 320 receives its own specific modifier coefficient based on the emission characteristics of nearby pollution sources for that particular pollutant type.

The local traffic modifier 334 is a quantitative adjustment factor that accounts for the additional health impact from vehicular emissions within a specified radius of the geographic location 302, recognizing that vehicle traffic constitutes a major source of pollutants including nitrogen oxides, particulate matter, carbon monoxide, and volatile organic compounds that may not be fully captured in regional air quality monitoring data. The modifier is determined algorithmically from the traffic volume data 316 by computing vehicle emission contributions based on daily traffic volume, applying distance-based weighting models based on proximity to the geographic location 302 (with closer roadways receiving higher weights), applying vehicle-type based weighting models (where higher-emitting vehicles like semi-trucks receive higher weights than lower-emitting vehicles like motorcycles), and aggregating these traffic-related contributions into a composite modifier coefficient that reflects the cumulative impact of nearby vehicular emissions. In some cases, the outdoor deterministic algorithm(s) 318 may leverage the Environmental Protection Agency's Motor Vehicle Emission Simulator (MOVES) model to enhance the accuracy of vehicular emission calculations. The MOVES model is a comprehensive emissions modeling system that estimates pollution from mobile sources by incorporating factors such as vehicle age distribution, fuel types, driving patterns, meteorological conditions, and emission control technology effectiveness. The local traffic modifier 334 is applied by multiplying the aggregated individual health impact scores 324 by the traffic modifier coefficient, thereby increasing the composite health impact score 136 to account for localized vehicular pollution exposure.

In variations, the outdoor deterministic algorithms 318 include an algorithm for calculating an outdoor composite health impact score 324 quantifying life expectancy loss, and multiple algorithms for calculating outdoor composite health impact scores 324 for sub-clinical health outcomes. To quantify life expectancy loss, the algorithm 318 determines each detected pollutant 320's individual health impact score 324 based on the pollutant 320's baseline concentration 322, response factor 138, independence factor 140, temporal weighting factor 142, atmospheric amplification factor 326, the pristine air baseline 328, and the duration 329 of exposure. In the life expectancy loss computation, however, the response factor 138 and the independence factor 140 are specific to all-cause mortality effects. For example, the response factor 138 is a coefficient that translates the baseline concentration 322 to a health impact metric quantifying life expectancy loss from all causes. Moreover, the independence factor 140 (extracted from the independence factor matrix 212 for quantifying life expectancy loss) may quantify the pollutant 320's individual effect on all-cause mortality given its combined exposure to other pollutants 320.

To quantify sub-clinical health outcomes, the algorithm 318 determines each detected pollutant 320's individual health impact score 324 based on the pollutant 320's baseline concentration 322, response factor 138, independence factor 140, temporal weighting factor 142, atmospheric amplification factor 326, the pristine air baseline 328, and the duration 329 of exposure. In the sub-clinical health outcome computation, however, the response factor 138 and the independence factor 140 are specific to the particular sub-clinical health outcome being evaluated. For example, the response factor 138 is a coefficient that translates the baseline concentration 322 to a health impact metric quantifying the impact on the specific sub-clinical health outcome. Moreover, the independence factor 140 (extracted from the independence factor matrix 212 for quantifying the particular sub-clinical health outcome) may quantify the pollutant 320's individual effect on that sub-clinical health outcome given its combined exposure to other pollutants 320.

Additionally or alternatively, the algorithms 318 may apply endpoint-specific factors to derive targeted health assessments from the composite health impact score. To determine a mortality-based health score, the algorithms 318 apply a mortality endpoint factor that isolates the portion of the composite score attributable to life expectancy loss and all-cause mortality effects, enabling quantification of fatal health outcomes from pollutant exposure. Similarly, the algorithms 318 may apply sub-clinical endpoint factors specific to various sub-clinical health outcomes to determine corresponding sub-clinical health scores, where each sub-clinical endpoint factor isolates the portion of the composite score attributable to a particular non-fatal health effect such as respiratory function decline, cardiovascular stress, cognitive impairment, or immune system suppression. These endpoint-specific factors are derived from epidemiological evidence and dose-response relationships that distinguish between fatal and non-fatal health impacts, allowing the system to provide users with both overall health impact assessment through the composite score and targeted health outcome predictions through endpoint-specific scoring that addresses mortality risk separately from quality-of-life impacts.

In various examples, the outdoor deterministic algorithm(s) 318 implement the following equation:

$$LEL = \sum ( BC \times RF \times PCTF \times IF \times TWF \times EF ) \times AAF$$

In the equation above, LEL represents the life expectancy loss, BC represents the baseline concentration 322 of a pollutant 320, RF is the response factor 138 of a pollutant 320, PCTF is the particle characterization toxicity factor 327 (of a particulate matter pollutant 320), IF is the independence factor 140 of a pollutant 320, TWF is the temporal weighting factor 142 of a pollutant 320, and EF is the endpoint factor that attributes health impacts to either all-cause mortality or sub-clinical health outcomes for a pollutant 320. These factors are summed over all pollutants 320 detected at the geographic location 302, with the atmospheric amplification factor, AAF, applied to the summation result. Although not shown in the equation, the local pollution source modifier 332 and the local traffic modifier 334 may further adjust the life expectancy loss. In some examples, the composite health impact score 336 may be generated by multiplying the life expectancy loss by a consistent scaling, thereby grounding the score 336 relatively to the life expectancy loss.

In one or more implementations, the algorithms 318 are configured to attribute portions of the outdoor composite health impact score 336 to various health domains based on the health domain distributions 218 of the detected pollutants 320. The algorithms 318 perform this attribution by multiplying each pollutant 320's individual health impact score 324 by its corresponding health domain distribution percentages, then aggregating these domain-specific contributions across all detected pollutants 320 to generate health domain scores, e.g., that sum to the total outdoor composite health impact score 336. For example, if nitrogen dioxide has an individual health impact score of 20 and a health domain distribution indicating 60% respiratory impact, 25% cardiovascular impact, and 15% neurological impact, the algorithm would attribute 12 points (20×0.60) to respiratory health, 5 points (20×0.25) to cardiovascular health, and 3 points (20×0.15) to neurological health. When combined with similar domain-specific attributions from other detected pollutants such as particulate matter and ozone, the algorithm generates comprehensive health domain scores that quantify the cumulative impact on respiratory function, cardiovascular health, neurological effects, and other physiological systems, providing users with detailed insights into which aspects of their health are most affected by the multi-pollutant exposure at the geographic location 302.

In alternative implementations, although the outdoor composite health impact score 336 is depicted and described as being generated using deterministic algorithms 318 in FIG. 3, the air quality analysis system 112 may be adapted to perform all or portions of the processing operations using machine learning approaches. This may be achieved by training the machine learning model 208 on the compendium of studies 204 and then specifically prompting the model on how to process the input data 304 to produce desired outputs such as the outdoor composite health impact score 336. In such implementations, the machine learning model 208 may receive prompts that include the outdoor air data history 306, terrain data 312, local pollution sources 144, traffic volume data 316, along with instructions directing the model to analyze these inputs and generate composite health impact scores based on the scientific knowledge encoded during training.

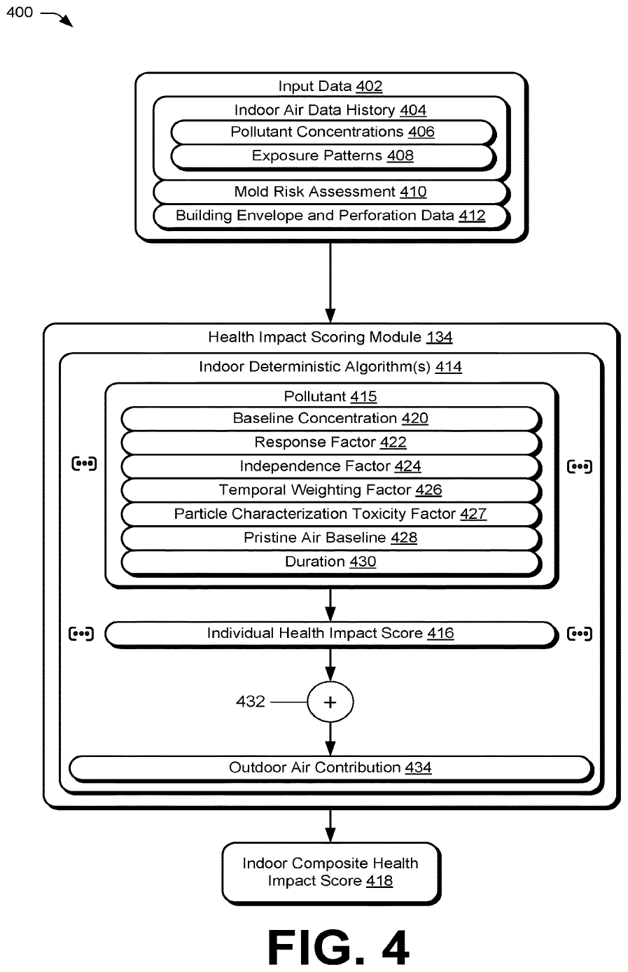

FIG. 4 depicts a system 400 in an example implementation showing operation of a health impact scoring module to leverage a deterministic algorithm during a runtime processing phase for composite health impact scoring of indoor air quality for multiple pollutants. In the system 400, the health impact scoring module 134 is illustrated as receiving a variety of input data 402. In particular, the input data 402 includes an indoor air data history 404 within an indoor environment of the structure 106, which includes pollutant concentrations 406 and exposure patterns 408. The indoor air data history 404 represents a comprehensive record of pollutant concentrations 406 within the structure 106 over an extended time period, e.g., two years. The pollutant concentrations 406 comprise measured levels of various airborne contaminants including particulate matter (PM2.5, PM10), volatile organic compounds, carbon dioxide, formaldehyde, and other pollutants measured by the indoor sensors 110 that impact indoor air quality within the structure 106. The exposure patterns 408 illustrate temporal variations in pollutant levels, showing how concentrations fluctuate throughout different time periods such as daily cycles, occupancy-related variations, HVAC system operation cycles, and temporary concentration spikes from indoor activities.

The indoor air data history 404 may be represented as time-series data comprising data points corresponding to specific temporal measurements of pollutant concentrations 406, thus capturing both the pollutant concentrations 406 and the exposure patterns 408. In some implementations, the indoor air data history 404 may be structured as a graph illustrating concentration levels at various points in time during the duration of evaluation. These points in time can be at any granularity such as daily, hourly, minutely, or even more frequent intervals depending on the monitoring capabilities and data collection frequency from the indoor sensors 110. The indoor air data history 404 is collected through the indoor sensors 110 deployed within the structure 106, which continuously monitor environmental conditions and transmit measurements to the air quality analysis system 112. For example, indoor air data 120 is periodically ingested by the air quality analysis system 112 and stored in the storage device 124, and the health impact scoring module 134 extracts the indoor air data history 404 from the storage device 124.

In one or more implementations, the input data 402 includes the mold risk assessment 410, further discussed above with reference to FIG. 1. The mold risk assessment 410 is an evaluation that focuses on the likelihood of and health impact due to mold contamination within the indoor environment, including a mold risk score that quantifies the likelihood of active mold sources contaminating the air of the indoor environment. The mold risk assessment 410 may be mold-species specific, where each mold species has its own health impact evaluation and mold risk score. The mold risk assessment 410 is generated by analyzing the mold lab test results 130 using machine learning models or deterministic algorithms that compare expected quantities of various mold species to observed quantities from the mold lab test results 130. The expected quantities may be determined by estimating a total air volume processed through an HVAC filter installed in the indoor environment using various techniques. By using the HVAC filter as a sample collection medium, the mold lab test results 130 identify (and the mold risk assessment 410 is grounded in) active mold sources that are currently releasing spores and mycotoxins into the air rather than surface mold from inactive sources.

In various implementations, the input data 402 includes building envelope and perforation data 412. The building envelope and perforation data 412 comprises information about the structure's air tightness, ventilation characteristics, and the rate at which outdoor air infiltrates into the indoor environment. Specific examples of this data include air changes per hour (ACH) measurements, blower door test results indicating building leakage rates, window and door sealing effectiveness ratings, construction material permeability coefficients, HVAC system specifications and operation parameters, structural integrity assessments that affect air flow, insulation quality and coverage data, and ventilation system capacity and efficiency metrics. Examples of online data sources 108 that may provide building envelope and perforation data 412 include online home listing services, government building code databases, energy efficiency certification programs, commercial building assessment platforms, architectural and engineering databases, property assessment records maintained by local municipalities, building performance monitoring services, and construction industry databases that maintain specifications for different building types and construction standards.

Generally, the health impact scoring module 134 is configured to process the input data 304 using one or more indoor deterministic algorithms 414, e.g., of the deterministic algorithms 220 output by the machine learning model 208 during the pre-processing stage. To do so, the outdoor deterministic algorithm(s) 414 analyze a plurality of pollutants 415 indicated by the indoor air data history 404 as present within the indoor environment of the structure 106. Notably, the pollutants 415 include mold, mold species, and other airborne pollutants, such as particulate matter, volatile organic compounds, carbon dioxide, formaldehyde, and other contaminants that impact indoor air quality within the structure 106. In particular, the indoor deterministic algorithm(s) 414 generate individual health impact scores 416 attributable to individual pollutants 415, and then aggregate the individual scores to generate the indoor composite health impact score 418. Generally, the indoor deterministic algorithm(s) 414 follow the same or similar processes to the outdoor deterministic algorithm(s) 318 to generate the indoor composite health impact score 418. Notably, the atmospheric amplification factor 326, the local pollution source modifier 332, and the local traffic modifier 334 are missing from the indoor computation as these parameters are specific to outdoor air.

Given a pollutant 415, for instance, the indoor deterministic algorithm 414 computes the pollutant 415's individual health impact score 416 by multiplying the baseline concentration 420 of the pollutant 415 to the pollutant 415's response factor 422. When the pollutant 415 is mold or a mold species, the baseline concentration 420 refers to the relative amount of the observed quantity from the mold lab test results 130 relative to the expected quantity determined from the processed air volume by the HVAC system in the structure 106 and particulate matter capture rate of the HVAC filter. For example, the baseline concentration 420 may be the amount by which the observed quantity exceeds the expected quantity. Moreover, the indoor deterministic algorithm 414 applies an independence factor 424 to the individual health impact score 416, where the independence factor 424 represents the pollutant 415's individual health impact given its combined exposure with other pollutants 415 in the indoor environment. The independence factors for primary and secondary contributors are determined in accordance with the techniques discussed above with reference to FIG. 3. The algorithm 414 also applies a temporal weighting factor 426 to the individual health impact score 416 based on the pollutant 415's exposure pattern 408, which accounts for variations in pollutant concentrations over time and adjusts the health impact calculation to reflect periods of elevated exposure, concentration spikes, and temporal variability patterns within the indoor environment.

In addition, the indoor deterministic algorithm(s) 414 generate and apply a particle characterization response factor 427 for particulate matter pollutants 415 in a similar manner to how the outdoor deterministic algorithm(s) 318 determine the particle characterization toxicity factor 327. As part of this, the mold risk assessment 410 includes a particle characterization analysis generated by various analysis techniques performed on the HVAC filter by the mold testing lab 128. The collected sample (e.g., the HVAC filter installed at the structure 106 for an installation period) may be analyzed using techniques such as chemical speciation via X-ray fluorescence spectroscopy for elemental composition, ion chromatography for ionic species identification, thermal-optical analysis for organic and elemental carbon fractionation, gas chromatography-mass spectrometry for organic molecular markers, biological characterization for endotoxins, allergens, and mycotoxins, and oxidential potential assays measuring capacity to generate reactive oxygen species.

The indoor sensors 110 may include multi-wavelength optical analyzers that measure particle size distributions across multiple size ranges, enabling differentiation between fine and coarse particulate matter fractions that exhibit different toxicity profiles. The sensors may employ laser-based light scattering and absorption measurements at multiple wavelengths to determine optical properties including scattering coefficients and absorption coefficients, which serve as indicators of particle composition and source characteristics. Through multi-wavelength analysis, the sensors may provide preliminary composition indicators that enable detection of combustion particles versus secondary aerosols based on their distinct optical signatures, where combustion-derived particles typically exhibit higher absorption coefficients due to their elemental carbon content, while secondary aerosols show predominantly scattering behavior. The continuous sensor data may be processed using algorithms that correlate optical properties with known particle types, enabling the system to estimate toxicity weighting factors in based on the predominant particle characteristics detected at each measurement interval.

The system 112 may implement a temporal fusion algorithm that combines the continuous sensor data with results of periodically conducted testing on the HVAC filter using the machine learning model 208 to estimate time-resolved particle composition between sampling intervals, thereby maintaining characterization continuity while controlling analytical costs. That is, the temporal fusion algorithm correlates the sensor measurements with the lab results to enhance accuracy in in particle characterization, chemical composition analysis, and emission source attribution. The temporal fusion algorithm enables source attribution via characterization by identifying particle origins through characteristic chemical fingerprints, distinguishing traffic emissions (characterized by hopanes and elemental carbon), biomass burning (identified by levoglucosan and potassium), secondary aerosols (marked by sulfate and nitrate), and biogenic particles (distinguished by endotoxins and fungal markers). By correlating these continuous sensor measurements with periodic laboratory analysis, the algorithm generates the particle characterization toxicity factors 427 that reflect the varying composition of particulate matter, enabling the system to apply composition-specific toxicity weightings to various particulate matter pollutants 415.

Like the outdoor calculation, the pollutant's individual health impact score 416 is grounded in its pristine air baseline 428, where the score 416 quantifies health impacts relative to if the occupant 118 were breathing pre-industrialization levels of air. The algorithm 414 applies a duration 430 (e.g., a time period over which the exposure to the pollutant is being evaluated) to the individual health impact score 416. As shown at 432, the individual health impact scores 416 for the pollutants 415 detected within the indoor environment of the structure 106 are aggregated.

In accordance with the described techniques, the algorithm(s) 414 are configured to apply an outdoor air contribution 434 to the aggregation result to arrive at the indoor composite health impact score 418. This involves, for example, computing an infiltration rate at which outdoor air infiltrates the indoor environment based on the building envelope and perforation data 412, and attributing a portion of the outdoor composite health impact score 336 to the indoor composite health impact score 418. The outdoor air contribution 434 represents the health impact attributable to outdoor pollutants that penetrate into the indoor environment through air leakage, ventilation systems, and building envelope permeability. The outdoor air contribution 434 is computed algorithmically by first determining the infiltration rate using the building envelope and perforation data 412, then computing the outdoor air contribution 434 based on the infiltration rate. In various implementations, for instance, the algorithm 414 multiplies the outdoor composite health impact score 336 by the calculated infiltration rate to determine what portion of the outdoor health impact should be attributed to the indoor environment. For example, if the outdoor composite health impact score 336 is 50 health impact units and the building envelope analysis indicates that 30% of outdoor air infiltrates into the indoor environment (infiltration rate of 0.3), the outdoor air contribution 434 would be 15 health impact units (50×0.3), which is then added to the health impacts from indoor-specific pollutants to generate the indoor composite health impact score 418.

Thus, the indoor composite health impact score 418 is a unified health impact metric that accounts for the (1) health impacts from indoor air data within the indoor structure detected by the indoor sensors 110, (2) health impacts from the mold risk assessment 410 as derived from the mold lab test results 130, and (3) health impacts from outdoor pollutants that infiltrate the indoor environment.

In one or more implementations, the air quality analysis system 112 may leverage the correlation between indoor and outdoor air quality data to provide diagnostic capabilities that identify the source of pollution affecting the indoor environment. By analyzing the concurrent time series data that captures both indoor air data 120 and outdoor air data 122 along a shared time axis, the system 112 (e.g., the machine learning model 208 and/or the deterministic algorithm(s) 220) may determine whether elevated indoor pollutant concentrations are primarily attributable to infiltration problems or internal pollution sources. When indoor pollutant levels correlate strongly with outdoor levels, this may indicate infiltration issues such as poor filtration systems, inadequate window sealing, or insufficient ventilation that allows outdoor pollution to penetrate the indoor environment. Conversely, when indoor pollutant concentrations remain elevated despite low outdoor levels or show patterns that diverge from outdoor trends, this may suggest the presence of internal pollution sources such as cooking activities, cleaning products, building materials, or mold infestations within the structure 106. This diagnostic capability may enable the system 112 to generate targeted recommendations for remediation strategies and communicating them to the client device 102 for display in a user interface. Examples, of these targeted recommendations include improving building envelope integrity and filtration systems for infiltration-related issues, or identifying and addressing specific indoor pollution sources for internally-generated contamination, thereby allowing occupants to take appropriate corrective actions based on the root cause of their air quality concerns.

In one or more implementations, the air quality analysis system 112 may perform correlation analysis to make inferences about relationships between different pollutant types, enabling more accurate identification and health impact assessment of specific contaminants within the indoor environment. For example, in mycotoxin analysis, the system 112 (e.g., the machine learning model 208 or the deterministic algorithm(s) 220) may implement multi-factor analysis by correlating various data inputs including mold lab test results 130 indicating elevated mold presence, high volatile organic compound (VOC) concentrations detected by the indoor sensors 110, and temporal humidity patterns (e.g., from the indoor sensors 110) showing VOC levels that correlate with moisture fluctuations within the structure 106. Through this correlation analysis, the system 112 may infer that detected VOCs are likely mycotoxins rather than other types of volatile organic compounds, as mycotoxins are secondary metabolites produced by certain mold species that typically exhibit concentration patterns aligned with moisture conditions and mold growth cycles. The system 112 may then leverage the specific mold species identified in the mold lab test results 130 to determine the exact type of mycotoxin present, such as aflatoxins, ochratoxins, or trichothecenes, which enables the health impact scoring module 134 to apply more precise response factors 138 and health domain distributions 218 specific to the identified mycotoxin rather than generic VOC parameters. This correlation-based identification process may result in adjusted composite health impact scores 136 that more accurately reflect the heightened health risks associated with mycotoxin exposure, which may have more severe health implications than other VOC sources such as cleaning products or building materials.

Like the outdoor air computation, the indoor deterministic algorithms 414, include an algorithm or calculating an indoor composite health impact score 418 quantifying life expectancy loss, and multiple algorithms for calculating indoor composite health impact scores 416 for various sub-clinical health outcomes. In accordance with the techniques discussed above with reference to the outdoor air computation, the algorithms 414 attribute portions of the indoor composite health impact score 418 to various health domains based on the health domain distribution 218 of the indoor pollutants 415. Moreover, although the indoor composite health impact score 418 is depicted and described as being generated using deterministic algorithms 414 in FIG. 4, the air quality analysis system 112 may be adapted to perform all or portions of the processing operations using machine learning approaches. This may be achieved by training the machine learning model 208 on the compendium of studies 204 and then specifically prompting the model on how to process the input data 402 to produce desired outputs such as the indoor composite health impact score 418.

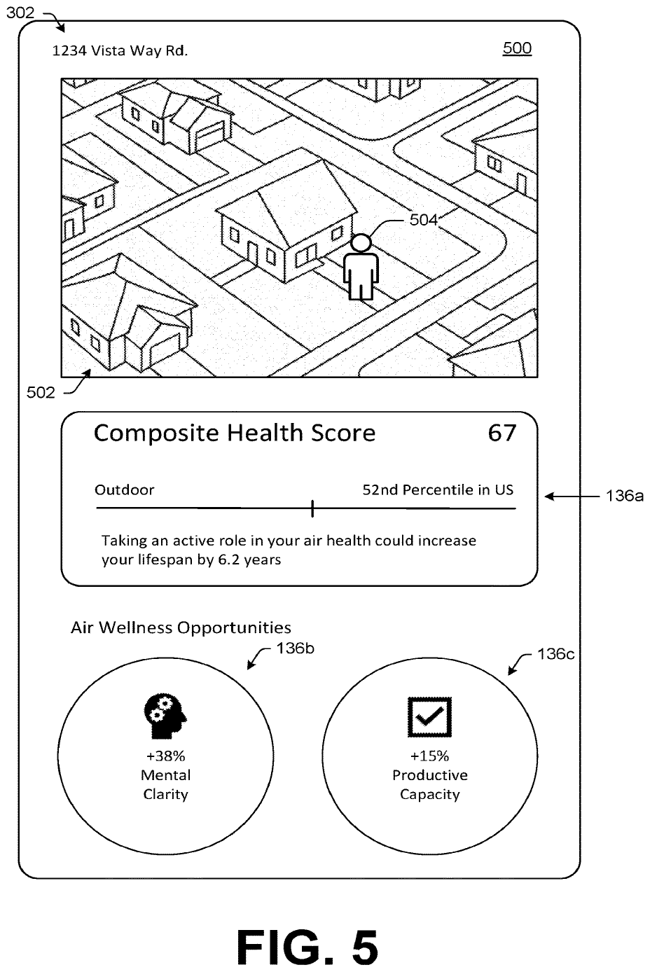

FIG. 5 depicts an example user interface 500 displaying composite health impact scores. As shown, the user interface 500 includes the geographic location 302, and a map view 502 of the geographic location 302. The map view 502 includes an indicator 504 of the geographic location 302 and/or structure 106 that is evaluated by the air quality analysis system 112. The user interface 500 includes a composite health impact score 136a quantifying life expectancy loss, and composite health impact scores 136b, 136c quantifying sub-clinical health outcomes. Here, the composite health impact score 136a is expressed in both a quantitative score (e.g., 67) and estimated life expectancy loss, e.g., 6.2 years. The user interface 500 also includes a slider element that shows where the composite health impact score 136a ranks within a nationwide distribution of composite health impact scores, e.g., the 52nd percentile. The composite health impact scores 136b, 136c are expressed in percentages relative to the pristine air baseline. For example, the occupant 118 can experience a 38% increase in mental clarity if the occupant 118 were breathing pollutant concentrations at their respective pristine air baselines, rather than the observed concentrations. Although the user interface 500 displays outdoor composite health impact scores 136a, 136b, 136c, a similar user interface can be generated that displays indoor composite health impact scores 136.

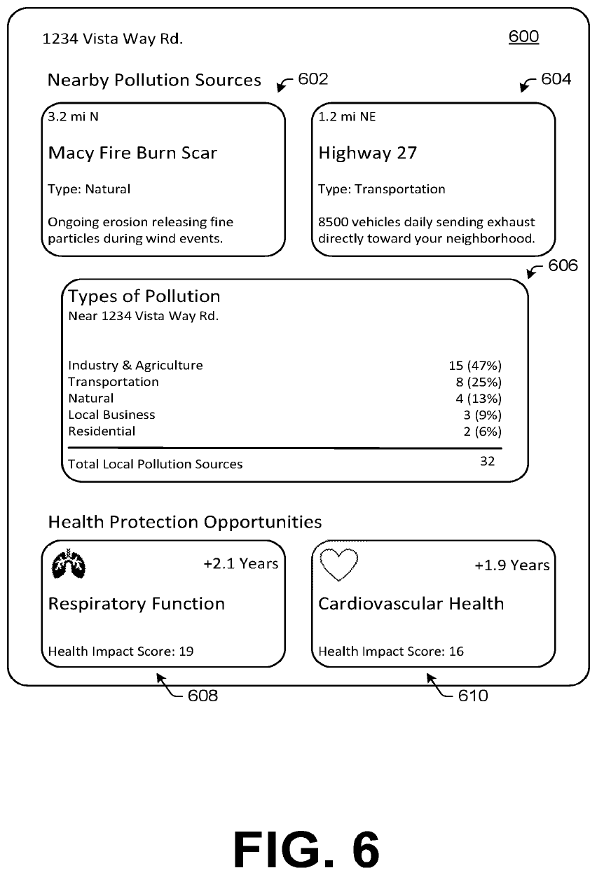

FIG. 6 depicts an example user interface 600 displaying local pollution sources and composite health impact scores attributable to health domains. As shown the user interface 600 include user interface elements 602, 604 describing local pollution sources 144 (e.g., within a radius of the geographic location 302). Each element 602, 604 includes a title of the local pollution source 144, a category of the local pollution source 144 (e.g., industry & agricultural, transportation, natural, local business, residential), and a distance from the geographic location 302. A user interface element 606 summarizes the distribution of the local pollution sources 144 within the radius of the geographic location 302 across the various categories. User interface elements 608, 610 include composite health impact scores 136 attributable to different health domains, e.g., respiratory function, and cardiovascular health. These scores may be expressed in quantitative scores, or estimated life expectancy loss.

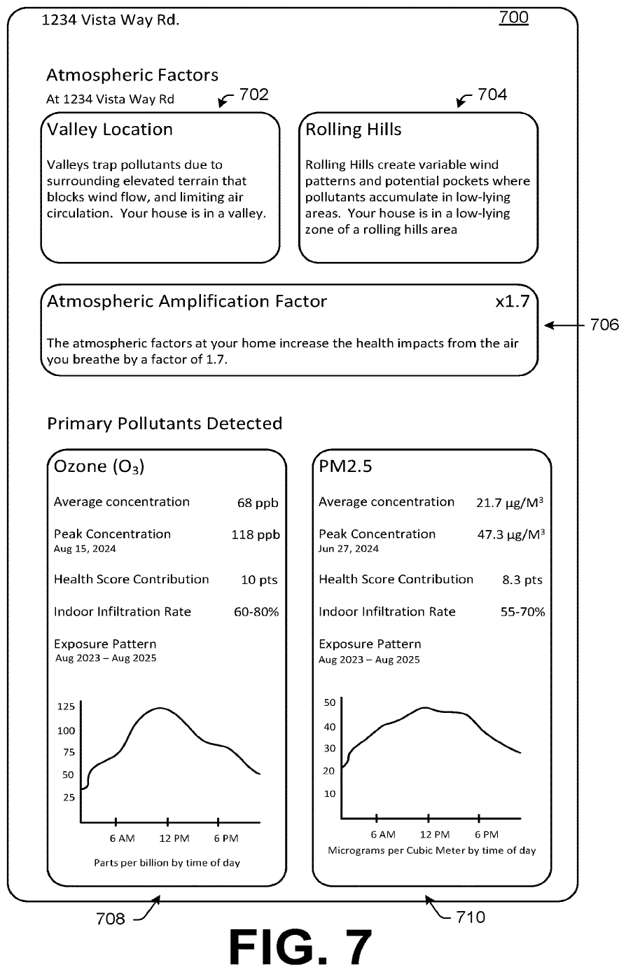

FIG. 7 depicts an example user interface 700 displaying a summary of atmospheric factors and detected pollutants. The user interface 700 includes user interface elements 702, 704 describing how various atmospheric factors (e.g., valley location and rolling hills) impact the composite health impact score 136 at the geographic location 302, as well as the atmospheric amplification factor 326, as shown at 706. In addition, the user interface 700 may include user interface elements (e.g., user interface elements 708, 710) summarizing concentration levels, exposure patterns, individual health impact scores 324, 416 of indoor or outdoor pollutants. For example, the user interface elements 708, 710 each include the average concentration, peak concentration, individual health impact score 324, and indoor infiltration rate of a particular pollutant, e.g., ozone and PM2.5. Moreover, each user interface element 708, 710 includes a graph showing a respective pollutant's daily concentration trends.

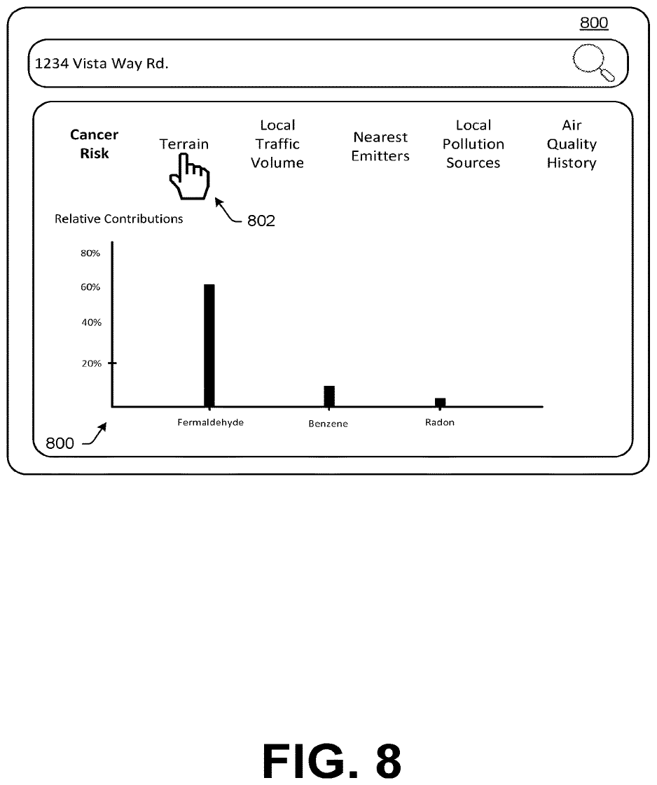

FIG. 8 depicts an example user interface 800 showing cancer-causing chemicals detected near a geographic location being evaluated for air quality. The user interface 800 includes a graph visualizing contributions of various cancer-causing chemicals of a census block containing the geographic location 302. This information may be obtained from online public data sources 108 such as databases and repositories from governmental monitoring agencies, like the EPA. The air quality analysis system 112 may retrieve this cancer risk data through APIs, process the information to identify specific carcinogenic compounds and their relative contributions to overall cancer risk at the geographic location, and then visualize and communicate this processed data to the client device 102 for display in the user interface 800. A user selection of “terrain” (e.g., as shown at 802) is effective to display a new user interface 900.

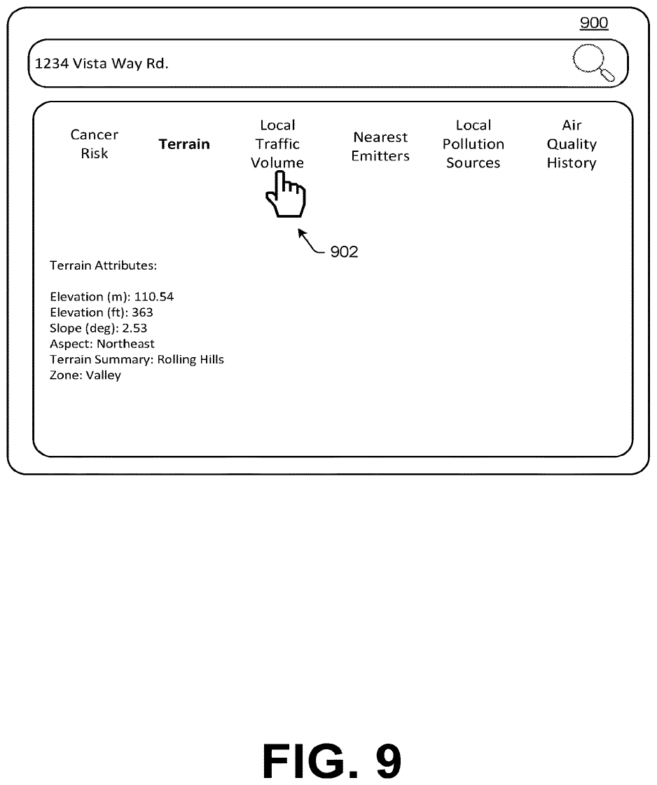

FIG. 9 depicts an example user interface 900 showing terrain data contributing to a composite health impact score at a geographic location. The user interface 900 includes terrain data 312 describing the geographic location 302's elevation, slope, aspect (e.g., the direction that the slope faces), a terrain summary (e.g., rolling hills), and a terrain zone, e.g., valley. The air quality analysis system 112 may retrieve this terrain data 312 from the online data sources 108 through APIs, process the information to extract relevant terrain data 312 that impacts the composite health impact score, and then visualize and communicate this processed data to the client device 102 for display in the user interface 900. A user selection of “local traffic volume” (e.g., as shown at 902) is effective to display a new user interface 1000.

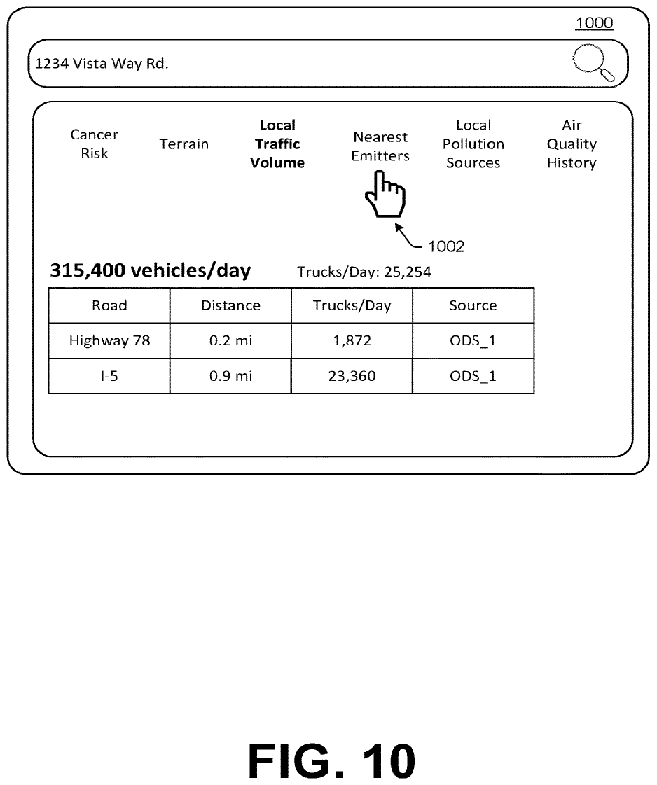

FIG. 10 depicts an example user interface 1000 showing traffic volume data contributing to a composite health impact score at a geographic location. The user interface 1000 includes traffic volume data 316 including local traffic statistics within a radius of the geographic location 302. The local traffic statistics may include a total number of vehicles passing within a radius of the geographic location per day (e.g., 315,400), and how many of those vehicles are trucks (e.g., 25,254). The local traffic statistics may identify multiple roadways (e.g., Highway 78 and I-5) that are within the radius of the geographic location 302, how close each roadway is to the geographic location 302, how many trucks travel along each roadway within the radius of the geographic location 302, and the online data source 108 that provides the traffic volume data 316 for each roadway. The air quality analysis system 112 may retrieve this traffic volume data 316 from the online data sources 108 through APIs, process the information to extract relevant traffic volume data 316, and then visualize and communicate this processed data to the client device 102 for display in the user interface 1000. A user selection of “nearest emitters” (e.g., as shown at 1002) is effective to display a new user interface 1100.

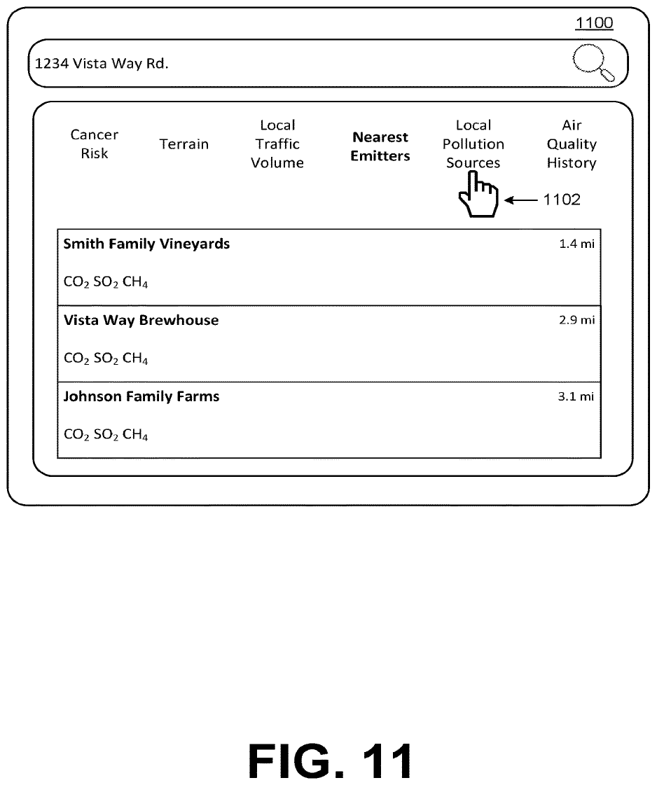

FIG. 11 depicts an example user interface 1100 showing registered emitters within a radius of a geographic location. The user interface 1100 includes a list of registered emitters (e.g., Smith Family Vineyards, Vista Way Brewhouse, and Johnson Family Farms), which are facilities that are officially documented and monitored by environmental regulatory agencies for their pollutant emissions. For each registered emitter, the user interface 1100 displays pollutants emitted by the registered emitter, such as carbon dioxide, sulfur dioxide, and methane for Smith Family Vineyards. User selection of an individual emitter may be effective to display a new user interface including emission metrics 314 for the selected emitter, such as reported annual emissions for each pollutant that the facility emits. The air quality analysis system 112 may retrieve this data from the online data sources 108 through APIs, process the information to extract relevant pollutants and/or emission metrics, and then visualize and communicate this processed data to the client device 102 for display in the user interface 1100. A user selection of “local pollution sources” (e.g., as shown at 1102) is effective to display a new user interface 1200.

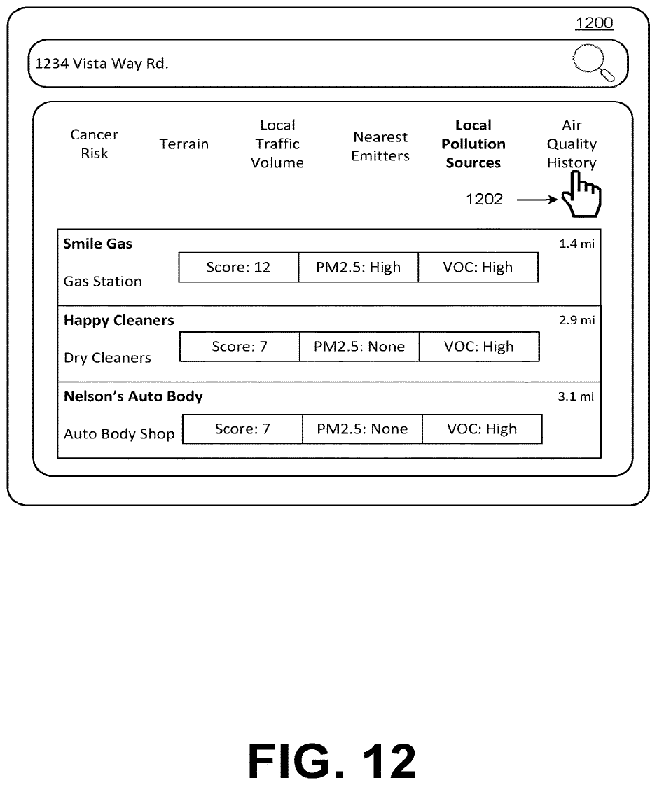

FIG. 12 depicts an example user interface 1200 showing local pollution sources within a radius of a geographic location. The user interface 1200 includes a list of local pollution sources 144, e.g., gas stations, dry cleaners, and auto body shops. As shown, the local pollution sources 144 include emission metrics 314, such as scores quantifying total emissions, and pollutant-specific classifications (e.g., low, medium, and high) describing emission characteristics of individual pollutants by respective sources 144. The local pollution sources 144 shown in FIG. 12 represent establishments categorized by establishment type with generalized emission metrics, whereas the registered emitters shown in FIG. 11 are facilities officially documented by environmental regulatory agencies with specific measured emission data. The air quality analysis system 112 may query online data sources 108 for establishments within a specified radius of the geographic location 302, then cross-reference these establishments against a local pollution source database to identify which qualify as pollution sources and retrieve their corresponding emission metrics 314. The system then visualizes and communicates this processed data to the client device 102 for display in the user interface 1200. A user selection of “air quality history” (e.g., as shown at 1202) is effective to display a new user interface 1300.

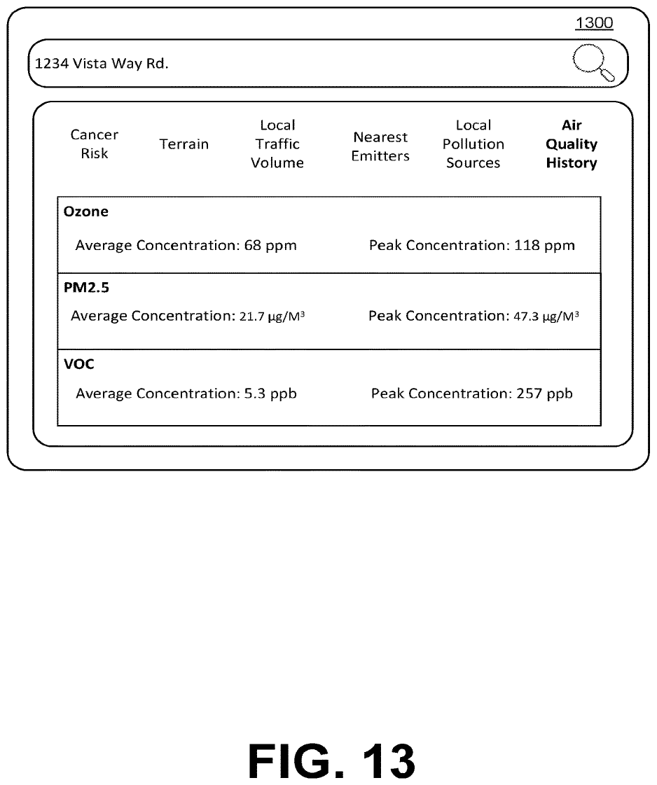

FIG. 13 depicts an example user interface 1300 showing an air quality history at a geographic location. The user interface 1300 includes a list of pollutants detected at the geographic location 302 (e.g., ozone, PM2.5, and VOCs). For each listed pollutant, the user interface 1300 includes concentration statistics, such as an average concentration and a peak concentration. User selection of an individual pollutant may be effective to display a more detailed view of the selected pollutant's concentration and exposure pattern. For example, upon selection of an individual pollutant, a new user interface may be displayed showing time-series data (e.g., a graph) comprising data points corresponding to temporal measurements of the pollutant's concentration over the duration of evaluation. The air quality analysis system 112 may retrieve this data from the online data sources 108 through APIs, process the information to extract relevant concentration and exposure pattern statistics, and then visualize and communicate this processed data to the client device 102 for display in the user interface 1300.

Having discussed exemplary details of composite health impact scoring of air quality for multiple pollutants, consider now some examples of procedures to illustrate additional aspects of the techniques.

### Example Procedure

This section describes an example procedure for composite health impact scoring of air quality for multiple pollutants. Aspects of the procedure may be implemented in hardware, firmware, or software, or a combination thereof. The procedure is shown as a set of blocks that specify operations performed by one or more devices and are not necessarily limited to the orders shown for performing the operations by the respective blocks.

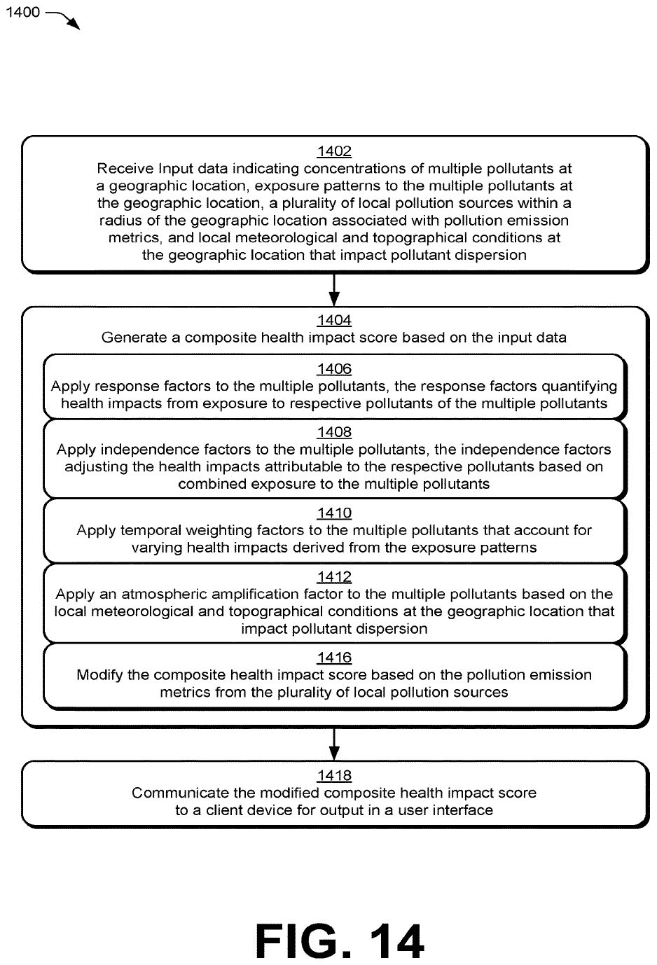

FIG. 14 is a flow diagram depicting an algorithm as a procedure 1400 in an example implementation that is performable by at least one processing device. In the procedure 1400, input data is received indicating concentrations of multiple pollutants at a geographic location, exposure patterns to the multiple pollutants at the geographic location, a plurality of local pollution sources within a radius of the geographic location associated with pollution emission metrics, and local meteorological and topographical conditions at the geographic location that impact pollutant dispersion (block 1402). By way of example, the health impact scoring module 134 receives input data 304 including the outdoor air data history 306 describing the pollutant concentrations 308 and the exposure patterns 310, the terrain data 312, and the local pollution sources 144. The input data may be received through application programming interfaces (APIs) that facilitate communication of the input data 304 between the air quality analysis system 112 and various online data sources 108. The outdoor air data history 306 represents time-series data including a comprehensive record of pollutant concentrations 308 at specific points in time (e.g., daily, hourly) over a duration of evaluation (e.g., two years) at the geographic location 302 to capture exposure patterns 310 that describe temporal variations in the pollutant concentrations 308. The terrain data 312 comprises geographical and topographical information about the physical landscape surrounding the geographic location, including elevation profiles and natural barriers that influence air flow patterns and pollutant dispersion. The local pollution sources 144 include facilities and establishments within a specified radius of the geographic location that emit pollutants, such as manufacturing plants, gas stations, and registered emitters documented by environmental regulatory agencies. The local pollution sources 144 are associated with emission metrics 314 that quantify the pollutant output from various sources, including numerical measurements and/or qualitative assessments of emission levels for different types of pollutants.

A composite health impact score is generated based on the input data (block 1404). For example, the health impact scoring module 134 leverages one or more deterministic algorithm(s) 318 to determine an outdoor composite health impact score 324 based on the input data 304. To generate the composite health impact score, response factors are applied to the multiple pollutants, where the response factors quantify health impacts from exposure to respective pollutants of the multiple pollutants (block 1406). By way of example, the deterministic algorithm(s) 318 apply response factors 138 to respective pollutants 320 indicated by the outdoor air data history 306 as present at the geographic location 302. The response factors 138 are quantitative coefficients that translate pollutant concentrations 322 into health impact metrics, with each response factor 138 being specific to a particular pollutant 320 and quantifying the expected health impact per unit of exposure. The response factors are applied by multiplying a baseline concentration 322 of each pollutant 320 by its corresponding response factor to calculate an individual health impact score 324 for that pollutant 320.

Independence factors are applied to the multiple pollutants, where the independence factors adjust the health impacts attributable to the respective pollutants based on combined exposure to the multiple pollutants (block 1408). By way of example, the deterministic algorithm(s) 318 apply independence factors 140 to the individual health impact scores 324 of respective pollutants 320. As part of this, an independence factor matrix 212 is generated by prompting the machine learning model 208 to analyze the compendium of scientific studies 204 and focus on identifying synergistic and antagonistic effects between pollutants, analyzing dose-response relationships for combined exposures, and quantifying the degree to which multiple pollutants affect overlapping physiological pathways. As a result, the independence factor matrix 212 is a structured table with a same set of pollutants along both axes, where individual cells contain independence factors quantifying the interaction effects between specific pollutant pairs.

Generally, independence factors 140 are quantitative coefficients that prevent overestimation of cumulative health impacts by accounting for overlapping biological pathways and synergistic effects when multiple pollutants are present simultaneously. A primary contributor among the detected pollutants 320 (e.g., the pollutant with the highest concentration relative to its pristine air baseline) receives an independence factor 140 of 1.0. Independence factors 140 for secondary contributors of the detected pollutants 320 are extracted from the independence factor matrix 212 by locating the cells corresponding to the combinations of secondary contributors with the primary contributor. The independence factors 140 of the secondary contributors are then weighted based on the concentrations of the detected pollutants 320 relative to their pristine air baselines. Application of the independence factors adjusts the individual health impact scores 324 of the secondary contributors to account for the combined exposure with the primary contributor.

Temporal weighting factors are applied to the multiple pollutants, where the temporal weighting factors account for varying health impacts derived from the exposure patterns (block 1410). For example, the deterministic algorithm(s) 318 apply temporal weighting factors 142 to the individual health impact scores 324 of respective pollutants 320. The temporal weighting factors 142 are determined by analyzing the exposure patterns 310 within the input data 304, including calculating variance in pollutant concentrations over the evaluation period, identifying frequency and magnitude of concentration spikes above baseline levels, and determining duration of elevated exposure periods. In some examples, since short bursts of elevated exposure may cause greater health impacts than sustained levels of lower exposure, the system may assign higher coefficients to pollutants 320 with greater concentration variability and more frequent exposure spikes. The temporal weighting factors 142 are applied by multiplying the individual health impact scores 324 by the corresponding temporal weighting coefficients, thereby adjusting the health impact calculations to reflect the increased health significance of variable exposure patterns and temporary concentration spikes compared to sustained exposure at consistent levels.

An atmospheric amplification factor is applied to the multiple pollutants based on the local meteorological and topographical conditions at the geographic location that impact pollutant dispersion (block 1412). For example, the deterministic algorithm(s) 318 apply an atmospheric amplification factor 326 to the individual health impact scores 324 of respective pollutants 320. The atmospheric amplification factor 326 represents a quantitative coefficient that adjusts pollutant concentration measurements to account for local meteorological and topographical conditions that influence air flow patterns, pollutant dispersion, and accumulation effects at the specific geographic location 302. The deterministic algorithm(s) 318 determine the atmospheric amplification factor 326 by analyzing terrain data 312 including elevation profiles, valley configurations, coastal proximity, urban canyon effects, prevailing winds, and natural barriers, then calculating how these topographical features impact air circulation and pollutant retention at the geographic location 302. Generally, the deterministic algorithm(s) 318 may assign a greater atmospheric amplification factor 326 to a geographic location 302 associated with topographical features that trap or concentrate pollutants (e.g., valleys surrounded by mountains or urban areas with limited air circulation), while assigning a lower atmospheric amplification factor 326 to locations with favorable dispersion conditions, e.g., coastal areas with consistent wind patterns or elevated terrain that facilitates pollutant dilution. The atmospheric amplification factor 326 is applied by multiplying the individual health impact scores 324 by the amplification coefficient, thereby adjusting the health impact calculations to reflect the actual pollutant exposure conditions influenced by local geography.

The composite health impact score is modified based on the pollution emission metrics from the plurality of local pollution sources (block 1416). For example, the individual health impact scores 324 of the detected pollutants are aggregated, and the deterministic algorithm(s) 318 modify the aggregation result based on the emission metrics 314 from the local pollution sources 144 to generate the outdoor composite health impact score 336. To do so, deterministic algorithm(s) 318 calculate distance-weighted contributions for the local pollution sources 144 based on their associated emission metrics 314 and proximity to the geographic location 302, with closer pollution sources receiving higher weights. The deterministic algorithm(s) 318 aggregate these contributions into a local pollution source modifier 332 that reflects the cumulative impact of the local pollution sources 144. The local pollution source modifier 332 is then applied by multiplying the aggregated individual health impact scores 324 by the modifier coefficient, thereby increasing the composite health impact score 136 to account for localized pollution exposure that may not be fully reflected in broader regional air quality measurements.

The modified composite health impact score is communicated to a client device for output in a user interface (block 1418). For example, the service provider system 104 communicates the outdoor composite health impact score 336 to the client device 102 over the network(s) 114. In response, the client device 102 displays the outdoor composite health impact score 336 in a user interface.

Having described an example of a procedure in accordance with one or more implementations, consider now an example of a system and device that can be utilized to implement the various techniques described herein.

### Example System and Device

FIG. 15 illustrates an example of a system generally at 1500 that includes an example of a computing device 1502 that is representative of one or more computing systems and/or devices that may implement the various techniques described herein. This is illustrated through inclusion of the application 116 and the air quality analysis system 112. The computing device 1502 may be, for example, a server of a service provider, a device associated with a client (e.g., a client device), an on-chip system, and/or any other suitable computing device or computing system.

The example computing device 1502 as illustrated includes a processing system 1504, one or more computer-readable media 1506, and one or more I/O interfaces 1508 that are communicatively coupled, one to another. Although not shown, the computing device 1502 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1504 is representative of functionality to perform one or more operations using hardware. Accordingly, the processing system 1504 is illustrated as including hardware elements 1510 that may be configured as processors, functional blocks, and so forth. This may include implementation in hardware as an application specific integrated circuit or other logic device formed using one or more semiconductors. The hardware elements 1510 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1506 is illustrated as including memory/storage 1512. The memory/storage 1512 represents memory/storage capacity associated with one or more computer-readable media. The memory/storage 1512 may include volatile media (such as random-access memory (RAM)) and/or nonvolatile media (such as read only memory (ROM), Flash memory, optical disks, magnetic disks, and so forth). The memory/storage 1512 may include fixed media (e.g., RAM, ROM, a fixed hard drive, and so on) as well as removable media (e.g., Flash memory, a removable hard drive, an optical disc, and so forth). The computer-readable media 1506 may be configured in a variety of other ways as further described below.

Input/output interface(s) 1508 are representative of functionality to allow a user to enter commands and information to computing device 1502, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1502 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1502. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1502, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1510 and computer-readable media 1506 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1510. The computing device 1502 may be configured to implement particular instructions and/or functions corresponding to the software and/or hardware modules. Accordingly, implementation of a module that is executable by the computing device 1502 as software may be achieved at least partially in hardware, e.g., through use of computer-readable storage media and/or hardware elements 1510 of the processing system 1504. The instructions and/or functions may be executable/operable by one or more articles of manufacture (for example, one or more computing devices 1502 and/or processing systems 1504) to implement techniques, modules, and examples described herein.

The techniques described herein may be supported by various configurations of the computing device 1502 and are not limited to the specific examples of the techniques described herein. This functionality may also be implemented all or in part through use of a distributed system, such as over a “cloud” 1514 via a platform 1516 as described below.

The cloud 1514 includes and/or is representative of a platform 1516 for resources 1518. The platform 1516 abstracts underlying functionality of hardware (e.g., servers) and software resources of the cloud 1514. The resources 1518 may include applications and/or data that can be utilized while computer processing is executed on servers that are remote from the computing device 1502. Resources 1518 can also include services provided over the Internet and/or through a subscriber network, such as a cellular or Wi-Fi network.

The platform 1516 may abstract resources and functions to connect the computing device 1502 with other computing devices. The platform 1516 may also serve to abstract scaling of resources to provide a corresponding level of scale to encountered demand for the resources 1518 that are implemented via the platform 1516. Accordingly, in an interconnected device embodiment, implementation of functionality described herein may be distributed throughout the system 1500. For example, the functionality may be implemented in part on the computing device 1502 as well as via the platform 1516 that abstracts the functionality of the cloud 1514.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method comprising:
receiving, by at least one processing device, input data indicating concentrations of multiple pollutants at a geographic location;
generating, by the at least one processing device, a composite health impact score based on the input data by:
applying response factors to the multiple pollutants, the response factors quantifying health impacts from exposure to respective pollutants of the multiple pollutants; and
applying independence factors to the multiple pollutants, the independence factors adjusting the health impacts attributable to the respective pollutants based on combined exposure to the multiple pollutants; and
communicating, by the at least one processing device, the composite health impact score to a client device for output in a user interface.

2. The method of claim 1, wherein applying the independence factors is effective to account for shared biological pathways impacted by the multiple pollutants to reduce overestimation of the health impacts from the combined exposure to the multiple pollutants.

3. The method of claim 1, further comprising:
prompting, by the at least one processing device, a machine learning model to generate an independence factor matrix based on one or more scientific studies, the independence factor matrix quantifying health impacts from combined exposure to different combinations of pollutants; and
extracting, by the at least one processing device, the independence factors from the independence factor matrix.

4. The method of claim 1, wherein generating the composite health impact score further includes applying an atmospheric amplification factor to the multiple pollutants based on local meteorological and topographical conditions at the geographic location that impact pollutant dispersion.

5. The method of claim 1, wherein the input data further includes exposure patterns to the multiple pollutants indicating variations in the concentrations during a duration of exposure, and generating the composite health impact score further includes applying temporal weighting factors to the multiple pollutants that account for varying health impacts derived from the exposure patterns.

6. The method of claim 1, wherein the composite health impact score is based on the concentrations of the multiple pollutants, and the concentrations are measured relative to a pristine air baseline representing pre-industrial atmospheric conditions.

7. The method of claim 1, wherein the input data includes a plurality of local pollution sources within a radius of the geographic location associated with pollution emission metrics, and generating the composite health impact score further includes modifying the composite health impact score based on the pollution emission metrics from the plurality of local pollution sources.

8. The method of claim 1, wherein the input data includes traffic data indicating a number of vehicles that travel within a radius of the geographic location, and generating the composite health impact score further includes modifying the composite health impact score based on the number of vehicles.

9. The method of claim 1, wherein generating the composite health impact score includes applying a particle characterization toxicity factor to a particulate matter pollutant of the multiple pollutants, and the particle characterization toxicity factor is based on varying health impacts from different particulate matter emission sources and chemical compositions.

10. The method of claim 1, further comprising:
during a pre-processing stage:
inputting, by the at least one processing device, a compendium of studies to a machine learning model, the compendium of studies describing health impacts from exposure to a plurality of pollutants; and
prompting, by the at least one processing device, the machine learning model to generate, based on the compendium of studies, an output including a deterministic algorithm for generating the composite health impact score as well as response factors and independence factors for the plurality of pollutants;
during a runtime processing stage:
extracting, by the at least one processing device, the response factors and the independence factors for the multiple pollutants from the output; and
generating, by the at least one processing device, the composite health impact score using the deterministic algorithm based on the response factors and the independence factors for the multiple pollutants.

11. The method of claim 1, wherein the input data includes the concentrations of the multiple pollutants in an outdoor environment at the geographic location, indoor air data collected by a plurality of sensors within an indoor environment of a structure at the geographic location, results of one or more laboratory mold tests conducted on a sample collected from the indoor environment, and building envelope and perforation data of the structure, wherein the indoor air data and the results of one or more laboratory mold tests indicate concentrations of indoor pollutants at the structure.

12. The method of claim 11, wherein generating the composite health impact score includes:
generating an outdoor composite health impact score based on the input data by applying the response factors and the independence factors to the multiple pollutants in the outdoor environment; and
generating an indoor composite health impact score based on the input data by:
applying the response factors and the independence factors to the indoor pollutants in the indoor environment; and
attributing a portion of the outdoor composite health impact score to the indoor composite health impact score based on an indoor infiltration rate derived from the building envelope and perforation data.

13. The method of claim 1, wherein the composite health impact score quantifies life expectancy loss or impacts on sub-clinical health outcomes that do not impact mortality.

14. The method of claim 1, further comprising attributing portions of the composite health impact score to multiple physiological domains including two or more of cardiovascular system, respiratory system, cancer risk, immune system, metabolic system, and neurological system.

15. A non-transitory computer-readable medium storing instructions that, when executed by one or more processors, cause the one or more processors to perform operations comprising:
receiving input data indicating concentrations of multiple pollutants at a geographic location and exposure patterns indicating variations in the concentrations during a duration of exposure;
generating a composite health impact score based on the input data by:
applying response factors to the multiple pollutants, the response factors quantifying health impacts from exposure to respective pollutants of the multiple pollutants; and
applying temporal weighting factors to the multiple pollutants that account for varying health impacts derived from the exposure patterns; and
communicating the composite health impact score to a client device for output in a user interface.

16. The non-transitory computer-readable medium of claim 15, wherein generating the composite health impact score further includes applying independence factors to the multiple pollutants, the independence factors adjusting the health impacts attributable to the respective pollutants based on combined exposure to the multiple pollutants.

17. The non-transitory computer-readable medium of claim 15, wherein generating the composite health impact score further includes applying an atmospheric amplification factor to the multiple pollutants based on local meteorological and topographical conditions at the geographic location that impact pollutant dispersion.

18. The non-transitory computer-readable medium of claim 15, wherein the input data includes a plurality of local pollution sources within a radius of the geographic location associated with pollution emission metrics, and generating the composite health impact score further includes modifying the composite health impact score based on the pollution emission metrics from the plurality of local pollution sources.

19. A system comprising:
one or more processors; and
a memory storing instructions that, when executed by the one or more processors, cause the system to perform operations comprising:
receiving input data indicating concentrations of multiple pollutants at a geographic location and a plurality of local pollution sources within a radius of the geographic location associated with pollution emission metrics;
generating a composite health impact score based on the input data by applying response factors to the multiple pollutants, the response factors quantifying health impacts from exposure to respective pollutants of the multiple pollutants;
modifying the composite health impact score based on the pollution emission metrics from the plurality of local pollution sources; and
communicating the modified composite health impact score to a client device for output in a user interface.

20. The system of claim 19, wherein the input data further includes exposure patterns to the multiple pollutants indicating variations in the concentrations during a duration of exposure, and generating the composite health impact score further includes:
applying independence factors to the multiple pollutants, the independence factors adjusting the health impacts attributable to the respective pollutants based on combined exposure to the multiple pollutants;
applying an atmospheric amplification factor to the multiple pollutants based on local meteorological and topographical conditions at the geographic location that impact pollutant dispersion; and
applying temporal weighting factors to the multiple pollutants that account for varying health impacts derived from the exposure patterns.
